(19) United States
(12) Patent Application Publication
(10) Pub. No.: US 2026/0102411 A1
(43) Pub. Date: Apr. 16, 2026

(54) USE OF INCYCLINIDE SYNERGIZED WITH COLISTIN IN PREPARATION OF BACTERICIDAL DRUG

(71) Applicant: Zhejiang University, Hangzhou (CN)

(72) Inventors: Ning Dong, Hangzhou (CN); Chen Xu, Zhenjiang (CN)

(21) Appl. No.: 19/418,939

(22) Filed: Dec. 13, 2025

(30) Foreign Application Priority Data
Mar. 28, 2025 (CN) 202510379597.3

(51) Int. Cl. A61K 31/65; A61K 38/12; A61P 31/04

(52) U.S. Cl. CPC A61K 31/65; A61K 38/12; A61P 31/04

(57) ABSTRACT

The present disclosure discloses use of incyclinide synergized with colistin in preparation of a bactericidal drug for an Enterobacteriaceae bacterium carrying an mcr gene. Incyclinide (Ini) is a chemically modified tetracycline derivative. The present disclosure demonstrates that Ini can restore the bactericidal effect of colistin against the Enterobacteriaceae bacterium carrying the mcr gene in vitro and in animal models. Mechanism analysis shows that Ini restores the pharmaceutical effect of colistin by inhibiting lipid A modification, inducing oxidative damage, and dissipating proton motive forces (PMF). Ini, as a colistin adjuvant, has great potential in combating the Enterobacteriaceae bacterium carrying the mcr gene.

### TECHNICAL FIELD

The present disclosure belongs to the technical field of bactericidal drug development, and in particular relates to use of incyclinide synergized with colistin in preparation of a bactericidal drug for an Enterobacteriaceae bacterium carrying an mcr gene.

### BACKGROUND

The widespread application of antibiotics in the medical field has greatly reduced infection-related mortality and incidence, bringing about revolutionary changes to healthcare. However, the overuse and misuse of antibiotics in human medicine and agriculture has created selection pressures, which promote the widespread spread of drug-resistant bacteria, greatly exacerbating the escalating crisis of antimicrobial resistance (AMR). The AMR not only threatens the health of individual patients, but also constitutes a global health crisis that may reverse decades of medical progress, highlighting the urgent need for coordinated global action led by the World Health Organization. In recent years, the AMR has spread rapidly, especially resistance to last-line antibiotics such as colistin, tigecycline, carbapenems, and cephenol.

Colistin (also known as polymyxin E) is a polypeptide cationic antibiotic that exhibits significant antibacterial activity against most gram-negative pathogens, particularly Pseudomonas aeruginosa, Acinetobacter baumannii, Klebsiella pneumoniae, and Escherichia coli. For these bacterial species, once they exhibit multidrug resistance, colistin often becomes the only effective antibiotic option. Colistin exerts its antibacterial effect by binding to anionic lipid A residues of lipopolysaccharides (LPSs) in the outer membranes of the gram-negative pathogens through electrostatic interaction. This binding leads to increased cell membrane permeability, leakage of cellular contents, and ultimately lytic cell death. However, the clinical application of colistin is significantly limited by its side effects, especially nephrotoxicity and neurotoxicity, and meanwhile by the rapid emergence and spread of drug resistance mechanisms in pathogens. The acquisition of colistin resistance is primarily due to a reduction in the electrostatic interaction between colistin and the negatively charged residues of lipid A. This process is generally mediated by the binding of a cationic substituent (e.g. phosphoethanolamine (pEtN) or a 4-amino-4-deoxy-L-arabinose group) to a phosphate group of an LPS lipid A component. This modification is catalyzed by corresponding transferases EptA and ArnT, thereby reducing the overall negative charge on the bacterial cell surface. Previously, these LPS modifications are thought to result from chromosomal mutations that activate two-component regulatory systems PhoP-PhoQ and PmrA-PmrB which control the expression of eptA or arnT. Because the spread of the resistance to chromosomal colistin is limited, its clinical impact is primarily confined to outbreak events. However, horizontal transfer of mobile colistin-resistance (mcr) genes via plasmids has been identified as a key mechanism facilitating the rapid spread of drug resistance within a specie and among species. Multidrug-resistant strains, especially those carrying resistance genes against other last-line antibiotics, may coexist with the mcr gene, This trend of gene coexistence poses a serious challenge to clinical treatment because it may render existing antibiotic treatment regimens ineffective and accelerate the depletion of effective antibiotic resources.

Developing new therapeutic strategies using adjuvants to restore the sensitivity of resistant bacteria to existing antibiotics is a promising approach to extend the service life of well-studied and clinically validated drugs. Among the current synergistic therapies used clinically, a prominent example is the combined use of β-lactam antibiotics and β-lactamase inhibitors. In addition to using inhibitors that target specific proteins encoded by resistance genes, the utilization of adjuvants to inhibit key pathways can also restore the potency of antibiotics. For example, it has been found by studies that natural flavonoids can restore the potency of colistin by interfering with the iron balance of the bacteria. However, there are currently no commercial colistin adjuvants available for clinical use, highlighting the urgent need to develop potential colistin adjuvants.

### SUMMARY

In order to overcome the shortcomings and deficiencies of the prior art, an objective of the present disclosure is to provide use of incyclinide synergized with colistin in preparation of a bactericidal drug for an Enterobacteriaceae bacterium carrying an mcr gene.

The present disclosure is achieved by: using incyclinide synergized with colistin in the preparation of a bactericidal drug for the Enterobacteriaceae bacterium carrying the mcr gene.

The present disclosure overcomes the deficiencies of the prior art and provides use of incyclinide synergized with colistin in the preparation of a bactericidal drug for the Enterobacteriaceae bacterium carrying the mcr gene. Incyclinide (Ini) is a chemically modified tetracycline derivative. The present disclosure demonstrates that Ini can restore the bactericidal effect of colistin against the Enterobacteriaceae bacterium carrying the mcr gene in vitro and in animal models. Mechanism analysis shows that Ini restores the pharmaceutical effect of colistin by inhibiting lipid A modification, inducing oxidative damage, and dissipating proton motive forces (PMF). Ini, as a colistin adjuvant, has great potential in combating the Enterobacteriaceae bacterium carrying the mcr gene.

Preferably, the Enterobacteriaceae bacterium includes Escherichia coli P47 and Escherichia coli P80.

Compared with the shortcomings and deficiencies of the prior art, the present disclosure has the following beneficial effects: The present disclosure proves through in vitro experiments, zebrafish infection models and mouse infection models that Incyclinide (Ini) works synergized with colistin to eliminate the Enterobacteriaceae bacterium carrying the mcr gene. Mechanistic studies have shown that Ini inhibits mcr expression to inhibit lipid A modification, thereby promoting the entry of colistin into a bacterial cell membrane. Furthermore, Ini dissipates a proton motive force (PMF), thereby impairing the function of a PMF-dependent efflux pump and leading to the accumulation of colistin within the membrane. The accumulated colistin induces oxidative stress, exacerbating membrane damage and ultimately leading to cell death. Therefore, Ini has great potential to become a highly effective colistin adjuvant to effectively address the increasingly serious threat posed by the infection caused by a pathogen carrying the mcr gene.

### BRIEF DESCRIPTION OF THE DRAWINGS

The patent or application file contains at least one drawing executed in color. Copies of this patent patent application publication with color drawing(s) will be provided by the Office upon request and payment of the necessary fee.

FIG. 1(A)-FIG. 1(K) show an in vitro experiment of a synergistic effect of Ini and colistin on eliminating an Enterobacteriaceae bacterium carrying a mcr gene and inhibiting biofilm formation; where FIG. 1(A)-FIG. 1(B) are isobolograms showing the synergistic effect of Ini and colistin on the Enterobacteriaceae bacterium carrying the mcr gene, with the X axis and Y axis being FIC_{Ini} and FIC_{colistin}, respectively; FIG. 1(C) is a heat map of an FIC index of Ini and colistin against the Enterobacteriaceae bacterium carrying the mcr gene; FIG. 1(D)-FIG. 1(K) are time-kill curves of the Enterobacteriaceae bacterium carrying the mcr gene under treatment with Ini, colistin, and a combination thereof; all experiments are conducted in triplicates independently, and the data is expressed as mean±standard deviation (SD). In the figures, Ini represents incyclinide; and CT represents colistin; statistical analysis is performed using GraphPad Prism 9.0 software, and P is determined using an unpaired t test between two groups (ns represents not statistically significant, *P<0.05, **P<0.01, ***P<0.001, and ****P<0.0001).

FIG. 2(A)-FIG. 2(D) show the inhibition effects on of Ini and colistin on biofilm formation; where FIG. 2(A)-FIG. 2(C) show the biofilm formation of Escherichia coli P47 after treatment with Ini, colistin, and a combination thereof for 24, 48, and 72 hours; FIG. 2(D) shows CLSM imaging of the biofilm structure of Escherichia coli P47 after treatment with Ini, colistin, and a combination thereof for 5 days; all experiments are conducted in triplicates independently, and the data is expressed as mean±standard deviation (SD). In the figures, Ini represents incyclinide; and CT represents colistin; statistical analysis is performed using GraphPad Prism 9.0 software, and P is determined using an unpaired t test between two groups (ns represents not statistically significant, *P<0.05, **P<0.01, ***P<0.001, and ****P<0.0001).

FIG. 3(A)-FIG. 3(J) show a microdilution test of Ini and colistin on chessboard broth of the Enterobacteriaceae bacterium carrying the mcr gene; where a dark green region represents higher bacterial cell density, and the figure shows the mean OD value of biological replicates at a wavelength of 600 nm.

FIG. 4(A)-FIG. 4(L) show the synergistic bactericidal effect of a combination of Ini and colistin in mammalian cells and an animal infection model; where FIG. 4(A) shows a cytotoxicity result of Ini against Vero cells as determined using an MTT experiment, and the Vero cells are treated with Ini, colistin, and a combination thereof, respectively; FIG. 4(B)-FIG. 4(C) show the intracellular bacterial load of Escherichia coli P47 in Vero cells after treatment with Ini, colistin, and a combination thereof; FIG. 4(D)-FIG. 4(F) show the inflammatory response of RAW264.7 cells induced by bacterial LPS (1 μg/ml) in the presence of different concentrations of Ini, where after treatment with Ini, the levels of TNF-α, IL-1β, and IL-10 cytokines in a culture sample are quantitatively determined by ELISA analysis; FIG. 4(G) shows a schematic diagram of experimental schemes for two animal models; FIG. 4(H) shows the survival rate of 16 zebrafishes in each group infected with clinically isolated Escherichia coli P80 carrying the mcr gene and received treatment with Ini, colistin, and a combination thereof, respectively, and P values are determined by a Log-rank (Mantel-Cox) test; FIG. 4(I) shows the survival rate of 8 mice in each group infected with clinically isolated Escherichia coli P80 carrying the mcr gene and received treatment with Ini, colistin, and a combination thereof, respectively, and P values are determined by a Log-rank (Mantel-Cox) test; and FIG. 4(J)-FIG. 4(L) show the bacterial counts in the liver, spleen, and kidney of a mouse peritonitis-sepsis infection model. In the figures, Ini represents incyclinide; and CT represents colistin. all experiments are conducted in triplicates independently, and the data is expressed as mean±standard deviation (SD). statistical analysis is performed using GraphPad Prism 9.0 software, and P is determined using an unpaired t test between two groups (ns represents not statistically significant, *P<0.05, **P<0.01, ***P<0.001, and ****P<0.0001).

FIG. 5 shows histological analysis of main organs of mice, and in the figure, on day 7 after the mice are injected with Ini (64 mg/kg), colistin (1 mg/kg) and a compound preparation thereof, the main organs (liver, spleen and kidney) removed from the mice in each group are stained with hematoxylin and eosin (H&E).

FIG. 6(A)-FIG. 6(Q) show that Ini enhances the damage to the bacterial cell membrane of Escherichia coli P47 induced by colistin; where, FIG. 6(A) shows the morphological changes of bacteria after treatment with Ini, colistin and a combination thereof as observed by scanning electron microscopy; FIG. 6(B)-FIG. 6(C) show the membrane permeability of bacteria treated with Ini, colistin and a combination thereof as determined by SYTOX Green staining; FIG. 6(D)-FIG. 6(E) show the membrane permeability of bacteria treated with Ini, colistin and a combination thereof as determined by evaluating the activity of β-galactosidase; FIG. 6(F)-FIG. 6(G) show the membrane fluidity in the presence of Ini, colistin and a combination thereof as determined by Laurdan staining; FIG. 6(H)-FIG. 6(I) show the evaluation of bacterial ΔΨ after treatment with Ini or colistin alone and a combined therapy thereof; FIG. 6(J)-FIG. 6(K) show that Ini and/or colistin have no effect on the ΔpH of bacteria as determined by BCECF-AM staining; FIG. 6(L)-FIG. 6(M) show the intracellular ATP levels after treatment with Ini or colistin alone and a combination thereof; FIG. 6(N)-FIG. 6(O) show the activity of bacterial efflux pumps as detected by Nile red staining; FIG. 6(P) shows the increased accumulation of colistin in bacteria caused by Ini; FIG. 6(Q) shows the synergistic mechanism of Ini in restoring membrane damage caused by colistin; In the figures, Ini represents incyclinide; and CT represents colistin. All experiments are conducted in triplicates independently, and the data is expressed as mean±standard deviation (SD). Statistical analysis is performed using GraphPad Prism 9.0 software, and P is determined using an unpaired/test between two groups (ns represents not statistically significant, *P<0.05, **P<0.01, ***P<0.001, and ****P<0.0001).

FIG. 7(A)-FIG. 7(B) show that Ini and colistin can inhibit the motility of Escherichia coli P47; FIG. 7(A) shows motility analysis using a semi-solid agar plate containing Ini, colistin, and a combination thereof; and FIG. 7(B) shows the migration distance (centimeters) measured after the inoculated plate was incubated at 37° C.; in the figures, Ini represents incyclinide; and CT represents colistin. Statistical analysis is performed using GraphPad Prism 9.0, and a P value is calculated using an unpaired t test between two groups (ns represents not statistically significant; *P<0.05; **P<0.01; ***P<0.001; ****P<0.0001).

FIG. 8(A)-FIG. 8(F) show that Ini enhances the antibacterial effect of colistin by targeting flagella formation and motility of bacteria; FIG. 8(A) is a volcano plot of gene expression difference distribution, with the horizontal axis representing the fold change of expression level (fold change value) and the vertical axis representing the relevant statistical value, with the adjusted P value of less than 0.05 used as a threshold (Student's t-test) to determine the genes with significant expression differences (DEGs); FIG. 8(C) and FIG. 8(E) show the GO annotation analysis of DEGs and genes involved in the most significantly enriched pathways such as bacterial flagella and cell motility; FIG. 8(B) and FIG. 8(D) show the KEGG enrichment analysis of DEGs and genes involved in the most important enriched pathways such as flagellar assembly and bacterial chemotaxis; FIG. 8(F) shows the quantitative determination of the number of Escherichia coli P47 bound to macrophage RAW 264.7 cells after 1 hour of in vitro infection; In the figures, Ini represents incyclinide; and CT represents colistin; All experiments are conducted in triplicates independently, and the data is expressed as mean±standard deviation (SD); statistical analysis is performed using GraphPad Prism 9.0 software, and P is determined using an unpaired/test between two groups (ns represents not statistically significant, *P<0.05, **P<0.01, ***P<0.001, and ****P<0.0001).

FIG. 9(A)-FIG. 9(L) show that the synergistic effect of Ini and colistin exacerbates the oxidative damage of the cell membrane of Escherichia coli P47; where FIG. 9(A)-FIG. 9(B) show that Ini or combined use of it with colistin accelerates the TCA cycle, FIG. 9(C) shows the fold change of the mRNA expression level of DEGs in the TCA cycle through transcriptome analysis; FIG. 9(D) and FIG. 9(E) show the ROS levels of bacteria after treatment with Ini, colistin and a combination thereof; FIG. 9(F)-FIG. 9(G) show the determination of SOD activity after adding colistin, Ini and a combination thereof; FIG. 9(H)-FIG. 9(I) show the hydrogen peroxide content of bacteria under the action of Ini, colistin and a compound preparation thereof; FIG. 9(J) shows the determination of the intracellular iron content of bacteria after treatment with Ini, colistin or a combination thereof; FIG. 9(K) shows that Ini converts intracellular iron from a ferric form to a ferrous form; FIG. 9(L) shows the synergistic mechanism of Ini and colistin in inducing membrane oxidative damage; In the figures, Ini represents incyclinide; and CT represents colistin; all experiments are conducted in triplicates, and the data is expressed as mean±SD; Statistical analysis is performed using GraphPad Prism 9.0, and a P value is determined using an unpaired t test between two groups (ns represents not statistically significant; *P<0.05; **P<0.01; ***P<0.001; ****P<0.0001).

FIG. 10(A)-FIG. 10(H) show that Ini inhibits the modification of lipid A; where FIG. 10(A) and FIG. 10(B) are quantitative analyses of the expression level of MCR-1 using Western blotting; FIG. 10(C) shows the determination of LPS content in bacteria treated with different concentrations of Ini; FIG. 10(D) shows the negative ion MALDI-TOF mass spectra of purified lipid A and lipid A-pEtN from Escherichia coli P47 in the presence of different concentrations of Ini; FIG. 10(E) shows the quantitative analysis of the content of lipid A and lipid A-pEtN; FIG. 10(F) shows the binding of Escherichia coli P47 to the colistin membrane in the presence of Ini; FIG. 10(G) shows the change in the positive charge on the membrane surface of the Escherichia coli P47 after treatment with Ini; FIG. 10(H) shows the synergistic action mechanism of Ini in terms of inhibiting the modification of lipid A and promoting bacterial absorption of colistin into the cell membrane; In the figures, Ini represents incyclinide; and CT represents colistin; all experiments are conducted in triplicates, and the data is expressed as mean±SD; Statistical analysis is performed using GraphPad Prism 9.0, and a P value is determined using an unpaired t test between two groups (ns represents not statistically significant; *P<0.05; **P<0.01; ***P<0.001; ****P<0.0001).

FIG. 11(A)-FIG. 11(F) show a microdilution test of Ini and colistin on chessboard broth of the Enterobacteriaceae bacterium; where a dark green region represents higher bacterial cell density, and the figure shows the mean OD value of biological replicates at a wavelength of 600 nm.

FIG. 12 shows the synergistic antibacterial mechanism of Ini and colistin on the Enterobacteriaceae bacterium carrying the mcr gene; where Ini inhibits the modification of lipid A by inhibiting the expression of mcr, thereby promoting the bacterial absorption of colistin into the cell membrane (1); Ini can also dissipate a proton motive force, damage the efflux pumps that rely on the proton motive force, and cause colistin to accumulate in the cell membrane (2); and the increase in the intracellular colistin content leads to bacterial membrane damage, and the accumulated colistin induces oxidative stress, aggravates membrane damage, and ultimately leads to cell death (3).

### DETAILED DESCRIPTION OF THE EMBODIMENTS

In order to make the objectives, technical solutions and advantages of the present disclosure more apparent, the present disclosure will be further described in detail hereafter with reference to the accompanying drawings and examples. It should be understood that the specific examples described herein are only used for explaining the present disclosure, and are not used for limiting the present disclosure.

### I. MATERIALS AND METHODS

### 1. Strains and Reagents

All strains used in the present disclosure are shown in Table 1 below.

TABLE 1
Bacterial strains

| | | Colistin | | Mechanism | | |
|---|---|---|---|---|---|---|
| Bacterial | | MIC | Colistin | of colistin | | FIC |
| species | Strain | (μg/ml) | susceptibility | resistance | Source | value |
| E. coli | P47 | 16 | Resistant | mcr-1 | conjugation | 0.16 |
| E. coli | P80 | 16 | Resistant | mcr-1 | clinical | 0.31 |
| E. coli | 57TC | 16 | Resistant | mcr-1, | clinical | 0.12 |
| | | | | mcr-8 | | |
| E. coli | LD22-1 | 8 | Resistant | mcr-1 | clinical | 0.25 |
| E. coli | LD67-1 | 8 | Resistant | mcr-1 | clinical | 0.19 |
| E. coli | LD93-1 | 8 | Resistant | mcr-1 | clinical | 0.28 |
| K. | P13 | >32 | Resistant | mcr-1, | clinical | 0.094 |
| pneumoniae | | | | mcr-8 | | |
| K. | TER244 | >32 | Resistant | mcr-9 | clinical | 0.066 |
| pneumoniae | | | | | | |
| Ent. cloaceae | ECC-A6 | >32 | Resistant | mcr-9 | clinical | 0.062 |
| Ent. cloaceae | SXJ55 | >32 | Resistant | mcr-9 | clinical | 0.039 |
| E. coli | BL21 | 8 | Resistant | mcr-1 | transformation | 0.19 |
| | MCR-1 | | | | | |
| E. coli | J53 | 2 | Sensitive | — | laboratory | 0.75 |

All strains were grown in Luria-Bertani (LB) broth or an LB agar plate at 37° C. overnight. Escherichia coli P47, which was derived from Escherichia coli J53 and carried an IncI2-type plasmid containing a mcr-1 gene, was selected as a model strain for mechanistic studies. Clinically isolated Escherichia coli P80 was used for in vivo evaluation. Ini (MedChem Express, Shanghai, China) was first dissolved in dimethyl sulfoxide (DMSO) (Meilunbio, Dalian, China) and then diluted in a culture medium to a desired working concentration.

### 2. Antibiotic Susceptibility Test

A broth dilution method was used for determining the minimum inhibitory concentration (MIC) of Ini and colistin on an Enterobacteriaceae bacterium carrying mcr-1, with reference to the guidelines of the Clinical and Laboratory Standards Institute (CLSI) (reference: CLSI. Performance Standards for Antimicrobial Susceptibility Testing, 34th ed. Clinical and Laboratory Standards Institute (2024)). Subsequently, the present disclosure used a chessboard analysis method to evaluate the synergistic effect of Ini and colistin in eliminating the Enterobacteriaceae bacterium carrying the mcr gene (reference: Xu, C. et al. Valnemulin restores colistin sensitivity against multidrug-resistant gram-negative pathogens. Communications Biology 7, 1122 (2024)). By calculating a fractional inhibitory concentration (FICI) index, the present disclosure drew contour maps and heat maps to intuitively display the interaction of the combination of Ini and colistin. The FIC index showed a synergistic effect.

### 3. Time-Dependent Killing Test

The synergistic antibacterial effect of Ini and colistin was determined by a time-dependent killing test, and the enterobacteria carrying the mcr gene in an exponential growth phase were diluted to 10⁶ CFU/ml. Then, bacterial cells were treated with sub-inhibitory concentrations of colistin, Ini, or a combination of both for 24 hours. At time points of 0, 3, 6, 18, and 24 hours, the survival cells were counted, and each group of experiments was conducted in triplicates. GraphPad Prism 9.0 (San Diego, CA, USA) was utilized to draw a curve showing a relationship between log₁₀ CFU/mL and time, so as to generate a time-killing curve.

### 4. Determination and Visualization of Biofilm Formation

A crystal violet staining method was utilized to detect the synergistic action of Ini and colistin in preventing biofilm formation. A bacterial suspension of Escherichia coli P47 (OD_{600 nm}=0.1) was cultured with a TSB medium containing sub-inhibitory concentrations of colistin, Ini, and a combination thereof at 37° C. for 24, 48, and 72 hours. After the end of the culture, the bacterial solution in the wells was discarded and the wells were washed with PBS. The biofilm was fixed with methanol, the methanol was discarded, and then the biofilm was washed again with PBS to remove the residual methanol. Then the biofilm was stained with addition of a 0.1% crystal violet solution, the staining solution was discarded, and the well plate was slowly rinsed to avoid washing away the biofilm. After the 96-well plate was naturally dried, it was added with 95% ethanol and shaken slowly to dissolve the crystal violet. After the end of the culture, the biofilm mass was quantified using a Tecan Spark multimode microplate reader (Tecan Australia Pty Ltd). Confocal laser scanning microscopy (CLSM) was employed to observe the effects of Ini and colistin. Overnight cultures were plated onto glass slides placed in a 24-well cell culture plates (BeyoGold™, China) and then treated with Ini, colistin, and a combination thereof at 37° C. for 5 days. After the end of the treatment, cells adhered to glass slides were washed and fixed, and then stained with PI for bacterial nucleic acids. The biofilm was imaged using CLSM (Leica, TCS SP8, Germany) with an excitation wavelength of 535 nm.

### 5. Cell Lines

RAW 264.7 cells, HeLa cells, and Vero cells were each cultured in a Dulbecco's modified Eagle's medium (DMEM, HyClone) supplemented with 10% (v/v) fetal bovine serum (HyClone).

### 6. Safety Evaluation

The cytotoxicity of Ini and colistin against mammalian cells was evaluated by an MTT assay (reference: Zeng, P. et al. Investigation of antibacterial activity, antibacterial activity, and mechanistic studies of an amphiphilic peptide against Acinetobacter baumannii. Biochimica et Biophysica Acta (BBA)-Biofilms 1863, 183600 (2021)). Briefly, Vero cells were cultured to a density of 8,000 cells per well. Then, the cells were co-cultured with Ini, colistin and a combination thereof for 24 hours. The cell viability was evaluated with a MTT cell proliferation and cytotoxicity assay kit (Beyotime, Shanghai, China) according to the instructions of the manufacturer. Finally, the absorbance was measured at the wavelength of 570 nm using a microplate reader to determine the cell viability.

The toxicity of Ini was further evaluated using BALB/c mice aged 5-8 weeks. The mice were divided into four groups and intravenously injected with Ini, colistin, and a compound preparation thereof, respectively. The control group was given the same volume of the solvent. Seven days later, mice were euthanized and dissected, and the spleens, kidneys, and livers were removed and immediately fixed with 4% paraformaldehyde in phosphate buffer. Subsequently, the organs were embedded in paraffin and stained with hematoxylin-eosin (H&E), and finally the organ sections were observed using an optical microscope to evaluate the histopathological changes of each organ.

### 7. Determination of Intracellular Bacterial Loading

A cell infection model was employed to evaluate the efficacy of Ini and colistin in eliminating intracellular bacteria. Vero cells were plated into a 24-well plate at a density of 1×10⁵ cells/well, and cultured under conditions of 37° C. and 5% CO₂ overnight. The cells were co-cultured with Escherichia coli P47 during the exponential growth phase at a multiplicity of infection (MOI) of 100:1. After 1 hour of culture, the supernatant was removed and the cells were transferred into fresh media containing Ini, colistin, and a combination thereof for further culture. After 6 hours of culture, the cells were rinsed twice with sterile PBS and then cultured with a fresh culture medium added with meropenem (8 μg/mL) to eliminate extracellular bacteria. After incubated for 15 minutes, the cells were rinsed twice again with sterile PBS and lysed with 0.1% (v/v) Triton X-100 (Beyotime, China) to quantify the number of viable intracellular bacteria.

### 8. In Vitro Cell Adhesion Test

HeLa cells were plated into a 24-well plate at a density of 0.8-1×10⁵ cells/well, and cultured under conditions of 37° C. and 5% CO₂ overnight. Escherichia coli P47 was cultured in fresh LB broth at a proportion of 1:100 for approximately 2 hours until the OD_{600 nm} reached 0.5. Different concentrations of Ini and colistin were added into the cell culture medium. Then, the cells were co-cultured with Escherichia coli P47 at a multiplicity of infection of 100:1 for 1 hour. After 1 hour of culture, the cells were rinsed for three times with sterile PBS, and lysed in 0.2% (v/v) Triton X-100 for 20 minutes. GraphPad Prism 9.0 was utilized to conduct quantitative analysis of the viable bacteria.

### 9. In Vitro Cytokine Measurement

The effect of Ini on cytokine production by RAW264.7 cells was evaluated using a cytokine assay (reference: Harimoto, T. et al. A programmable encapsulation system improves delivery of therapeutic bacteria in mice. Nature Biotechnology 40, 1259-1269 (2022)). The RAW264.7 cells were cultured to a density of 2×10⁴ cells/well. The levels of TNF-α, IL-1β, and IL-10 in the cell culture supernatant were determined using an enzyme-linked immunosorbent assay (ELISA) kit (Boster Biological Technology, China), and the operating steps were conducted according to the instructions of the manufacturer.

### 10. Zebrafish Infection Model

A zebrafish infection model was utilized to evaluate the synergistic effect of Ini and colistin. Escherichia coli P80 bacterial cells in the exponential phase were rinsed and resuspended with sterile PBS. Adult zebrafishes were divided into 6 groups (16 per group), and intraperitoneally injected with 0.1 mL of 3.0×10⁹ CFU/mL Escherichia coli P80. At 1 hour after infection, the zebrafishes were intraperitoneally injected with different concentrations of Ini, colistin, and a combination thereof. The untreated zebrafishes injected with the solvent only served as a control group.

### 11. Mouse Infection Model

The synergistic effect on treating infection with the Enterobacteriaceae bacterium carrying the mcr gene was further evaluated using a mouse infection model (references: Cai, J. et al. Structural-Activity Relationship-Inspired the Discovery of Saturated Fatty Acids as Novel Colistin Enhancers. Advanced Science 10, 2302182 (2023), and Xu, C. et al. Imidazole Type Antifungal Drugs Are Effective Colistin Adjuvants That Resensitize Colistin-Resistant Enterobacteriaceae. Advanced Therapeutics 3, 2000084 (2020).). Briefly, BALB/c mice aged 5-8 weeks were randomly divided into 6 groups (8 mice per group). Subsequently, all mice were infected with clinically isolated Escherichia coli P80 carrying the mcr gene at an infection dose of 3×10⁸ CFU/ml to establish a sepsis infection model for further study. At 1 hour after infection, the mice were treated with Ini, colistin, and a compound preparation thereof, respectively. All animal experiments were approved by the Institutional Animal Care and Use Committee of Zhejiang University, and the present disclosure strictly complied with all relevant ethical regulations on animal use.

12. Detection with Scanning Electron Microscope (SEM)

The cell morphologies of the Enterobacteriaceae bacteria carrying mcr in the treatment with Ini, colistin and a compound preparation thereof were observed by scanning electron microscope imaging technology. (reference: Xu, C. et al. Bactericidal, anti-biofilm and anti-virulence activity of vitamin C against carbapenem-resistant hypervirulent Klebsiella pneumoniae. iScience 25, 103894 (2022)). The bacterial cells were rinsed twice with sterile PBS, and then fixed with 2.5% glutaraldehyde overnight. After completion of the fixation, the bacterial cells were centrifuged at 10,000 rpm for 1 minute and then dehydrated with 100% ethanol. Finally, the cell morphology was observed using a Hitachi SU8010 scanning electron microscope (Tokyo, Japan).

### 13. Determination of Cell Membrane Permeability

The synergistic effect of Ini and colistin on bacterial membrane permeability was determined using SYTOX Green (Thermo Fisher Scientific, Waltham, USA) (reference: Sochacki, K. A., Barns, K. J., Bucki, R. & Weisshaar, J. C. Real-Time Attack On Single Escherichia coli Cells By The Human Antimicrobial Peptide Ll-37. Proceedings of the National Academy of Sciences of the United States of America 108, E77-81, doi:10.1073/pnas.1101130108 (2011)). Briefly, the Escherichia coli P47 in the exponential phase was treated with various concentrations of Ini, colistin, and a combination thereof for 2 hours. After the treatment, the bacterial cells were collected by centrifugation and resuspended in sterile saline to an OD_{600 nm} of 0.2. The bacterial suspension was stained with SYTOX Green at a final concentration of 1 μM for 10 minutes. The fluorescence intensity of each test group was detected using a Tecan Spark multimode microplate reader.

### 14. Determination of β-Galactosidase Activity

Moreover, the bacterial membrane permeability during the treatment with Ini, colistin, and a combination thereof was further evaluated using a β-galactosidase activity assay (reference: Cai, J. et al. Structural-Activity Relationship-Inspired the Discovery of Saturated Fatty Acids as Novel Colistin Enhancers. Adv Sci (Weinh) 10, e2302182, doi:10.1002/advs.202302182 (2023).). The Escherichia coli P47 cultured overnight was diluted 100-fold with a fresh culture medium, and then continually cultured for 4 hours. Bacterial cells were collected by centrifugation and resuspended in sterile PBS to an OD_{600 nm} of 0.2. The bacterial suspension was added with O-nitrophenyl-β-D-galactopyranoside (ONPG) at a final concentration of 3 mM and then incubated at 37° C. for 1 hour. Finally, the absorbance was measured at a wavelength of 420 nm to evaluate β-galactosidase activity.

### 15. Determination of Membrane Fluidity

Membrane fluidity was evaluated using Laurdan staining (reference: Müller, A. et al. Daptomycin inhibits cell envelope synthesis by interfering with fluid membrane microdomains. Proceedings of the National Academy of Sciences 113, E7077-E7086 (2016).). The bacterial suspension of Escherichia coli P47 in the exponential phase was centrifuged and resuspended with sterile PBS. Then the cells were stained with laurdan with a final concentration of 10 μM under dark conditions at 37° C. for 30 minutes. The stained cell cultures were rinsed twice with PBS and then incubated with different concentrations of Ini, colistin, and a compound preparation thereof at 37° C. for 2 hours. Laurdan GP was utilized to evaluate membrane fluidity, and its calculation formula was: GP=(I435−I490)/(I435+I490).

### 16. Membrane Depolarization

The bacterial membrane potential (Δφ) after treatment with Ini and colistin was determined using DiSC₃ (5) fluorescence staining. The Escherichia coli P47 cells in the exponential phase were treated with different concentrations of Ini, colistin and a combination thereof for 2 hours. The cells were rinsed with a PBS buffer, then resuspended with KCl in PBS with a final concentration of 100 mM to an OD_{600 nm}=0.2, stained with DiSC₃ (5) at a final concentration of 1 μM, and incubated at 37° C. with protection from light. The fluorescence level was measured using a Tecan Spark multimode microplate reader with an excitation wavelength of 610 nm and an emission wavelength of 660 nm.

### 17. Proton Gradient (ΔpH)

The bacterial ΔpH was evaluated using BCECF-AM staining (reference: Worthan, S. B. et al. Evolution of pH-sensitive transcription termination in Escherichia coli during adaptation to repeated long-term starvation. Proceedings of the National Academy of Sciences 121, e2405546121 (2024).). The Escherichia coli P47 bacterial cells in the exponential phase were rinsed with PBS and then stained with BCECF-AM (Beyotime, Shanghai, China) at a final concentration of 10 μM under dark conditions for 30 minutes. After the end of staining, bacteria were treated with different concentrations of Ini, colistin, and a combination thereof for 10 minutes. The fluorescence intensity was monitored using a Tecan Spark multimode microplate reader with an excitation wavelength of 488 nm and an emission wavelength of 535 nm.

### 18. Intracellular Adenosine Triphosphate (ATP) Level

Escherichia coli P47 bacterial cells were centrifuged and resuspended in PBS (OD_{600 nm}=0.5). After 2 hours of treatment with Ini, colistin, and a combination thereof, the bacterial cells were collected by centrifugation and lysed. The ATP content in the supernatant was determined using an enhanced ATP assay kit (Beyotime, Shanghai, China) and a Tecan Spark multimode microplate reader (reference: Jia, Y. et al. Melatonin prevents conjugative transfer of plasmid-mediated antibiotic resistance genes by disrupting proton motive force. Pharmacological Research 175, 10⁵978 (2022).).

### 19. Swimming Test

The Escherichia coli P47 in the exponential phase was inoculated into a semi-solid LB agar medium (0.3% agar) containing different concentrations of Ini, colistin, and a combination thereof. After 24 hours of culture at 37° C., the formed swimming halos were imaged and their migration distances were measured. This method was utilized to evaluate the effects of Ini and colistin on the swimming motility of bacteria.

### 20. Nile Red Efflux Experiment

The Escherichia coli P47 bacterial cells in the stationary phase were centrifuged and resuspended in a sterile PBS buffer containing 1 mM MgCl₂ (OD_{600 nm}=1.0). The bacterial suspension was pre-incubated with CCCP, and subsequently treated with different concentrations of Ini, colistin, and a combination thereof. After treatment, the bacterial cells were stained with 5 μM Nile Red for 1 hour. The fluorescence in the bacterial suspension was monitored using a Tecan Spark multimode microplate reader for 7 minutes (reference: Bohnert, J. A., Karamian, B. & Nikaido, H. Optimized Nile Red efflux assay of AcrAB-TolC multidrug efflux system shows competition between substrates. Antimicrob Agents Chemother 54, 3770-3775, doi:10.1128/AAC.00620-10 (2010).).

### 21. Colistin Quantification

The Escherichia coli P47 bacterial cells were resuspended in sterile PBS containing 16 μg/ml of colistin (OD_{600 nm}=0.6) in the presence or absence of Ini, and incubated at 37° C. for 30 minutes. The cells were harvested by centrifugation, washed three times with sterile PBS, and then resuspended in sterile saline. The suspension was added with 200 μl of a glycine-hydrochloric acid buffer with a pH value of 2.4, and incubated for 30 minutes to dissociate colistin bound to the bacterial membrane. Subsequently, the bacterial cells were collected and lysed using ultrasonic waves to release intracellular colistin accumulated within the bacteria. The colistin content was quantified using a colistin enzyme-linked immunosorbent assay (ELISA) kit according to the instructions of the manufacturer.

### 22. Reactive Oxygen Species (ROS) Level

The intracellular ROS level was measured using a fluorescent probe 2′,7′-dichlorodihydrofluorescein diacetate (DCFH-DA) (reference: Xu, C. et al. Bactericidal, anti-biofilm, and anti-virulence activity of vitamin C against carbapenem-resistant hypervirulent Klebsiella pneumoniae. Iscience 25, 103894 (2022).). The Escherichia coli P47 bacterial cells in the Logarithmic phase were stained with 10 mM DCFH-DA at 37° C. for 30 minutes, washed three times, and resuspended with sterile PBS containing Ini, colistin, and a complex thereof. The fluorescence intensity was detected using a Tecan Spark multimode microplate reader with an excitation wavelength of 488 nm and an emission wavelength of 525 nm.

### 23. Superoxide Dismutase (SOD) Activity

The intracellular SOD activity was measured using a WST-8 total superoxide dismutase assay kit (S0101, Beyotime, China). The overnight cultured Escherichia coli P47 was diluted 100-fold and then continually cultured for 4 hours. The bacterial cells were harvested and resuspended in sterile PBS (OD_{600 nm}=0.5). Then, the cells were treated with Ini, colistin and a combination thereof for 2 hours. The intracellular SOD activity was measured according to the instructions of the manufacturer.

### 24. Quantification of NAD⁺/NADH Ratio

The Escherichia coli P47 bacterial cells in the exponential phase were cultured with Ini, colistin and a complex thereof for 2 hours. After the end of the culture, the bacterial cells were collected and resuspended in sterile PBS to OD_{600 nm}=0.2, and an NAD⁺/NADH detection kit (Beyotime, China) was utilized for NAD⁺/NADH detection.

### 25. Hydrogen Peroxide Content Test

The Escherichia coli P47 bacterial culture was cultured with Ini, colistin, and a combination thereof until OD_{600 nm} reached 0.5. Subsequently, the culture was harvested and homogenized with ultrasonic waves. The cell lysate was collected, and the hydrogen peroxide content was determined using a hydrogen peroxide detection kit (S0038, Beyotime, China) according to the instructions of the manufacturer.

### 26. Determination of Intracellular Iron Content

The Escherichia coli P47 was treated with colistin in the presence or absence of Ini for 2 hours, and the bacterial culture was harvested and homogenized with ultrasonic waves to obtain 10⁷ cells. The cell lysate was collected to determine the intracellular iron content. Total iron and the iron in the ferrous iron form were quantified by using a total iron colorimetric kit (E-BC-K139-M, Elabscience, USA) and a cell ferrous colorimetric kit (E-BC-K881-M, Elabscience, USA), respectively.

### 27. Transcriptome Analysis

Transcriptome analysis was performed on the Escherichia coli P47 exposed to 0.5 μg/mL of colistin in the absence of Ini or in the presence of 2 μg/mL of Ini (reference: Chen, C., Cai, J., Shi, J., Wang, Z. & Liu, Y. Resensitizing multidrug-resistant Gram-negative bacteria to carbapenems and colistin using disulfiram. Communications Biology 6, 810 (2023).). The total RNA was extracted using a bacterial RNA extraction kit (Vazyme, Nanjing, China) and sent to Shanghai Majorbio Bio-Pharm Technology Co., Ltd. (Shanghai, China) for sequencing on a Illumina Hiseq system. The reference for bioinformatics analysis methods was: Xu, C. et al. Valnemulin restores colistin sensitivity against multidrug-resistant gram-negative pathogens. Communications Biology 7, 1122 (2024).

### 28. Western Blot Analysis

The expression of mcr-1 in Escherichia coli BL21 cells transformed with a recombinant plasmid pET28a-mcr-1 (GenBank No.: KY283125.1) (hereinafter referred to as BL21/pMCR-1 for short) was evaluated (reference: Ye, X. et al. Peptide MSI-1 inhibited MCR-1 and regulated outer membrane vesicles to combat immune evasion of Escherichia coli. Microbial Biotechnology 16, 1755-1773 (2023).). BL21/pMCR-1 bacterial cells were cultured with different concentrations of Ini. After the end of the culture, the cells were lysed by ultrasonication and centrifuged at 12,000 g for 2 minutes to collect the supernatant rich in soluble MCR-1 proteins. The protein samples were loaded onto a 10% or 12% SDS-PAGE gels and subjected to Western blotting. The protein was developed with an enhanced chemiluminescence substrate and quantified with ImageJ software.

### 29. LPS Content Determination

The LPS content was determined by a Limulus Amebocyte Lysate (LAL) test (reference: Ma, Y. et al. Deletion of CD38 mitigates the severity of NEC in experimental settings by modulating macrophage-mediated inflammation. Redox Biology 77, 103336 (2024).). The overnight cultured Escherichia coli P47 was diluted 100-fold and then continually cultured for 4 hours. The bacterial cells were collected by centrifugation (5,000 rpm, 5 minutes) and resuspended in PBS to an OD_{600 nm}=0.5. After treatment with various concentrations of Ini for 2 hours, the LPS content in the bacterial cells was determined using a chromogenic LAL endotoxin detection kit (C0276S, Beyotime, Shanghai, China) according to the instructions of the manufacturer.

### 30. Membrane Charge

The membrane charge was determined by FITC-labeled poly-L-lysine (PLL) (reference: Cai, J. et al. Structural-Activity Relationship-Inspired the Discovery of Saturated Fatty Acids as Novel Colistin Enhancers. Advanced Science 10, 2302182 (2023).). After incubated with different concentrations of Ini until OD_{600 nm} reached 0.5, the bacterial cells were collected by centrifugation and washed twice with sterile saline. Subsequently, the cells were resuspended in an equal volume of a Tris buffer (0.03 M, pH 8.0 Tris, 20% sucrose, 20 μg/ml FITC), and incubated in the dark for 10 minutes. Then, the cells were washed three times with a Tris buffer (0.03 M, pH 8.0 Tris, 20% sucrose, without FITC). The fluorescence level of the sample was measured using a Tecan Spark multimode microplate reader at an excitation wavelength of 490 nm and an emission wavelength of 525 nm.

### 31. Extraction and Quantification of Lipid A

The Escherichia coli P47 was cultured in 200 ml of MH broth containing Ini (at a concentration range of 0-8 μg/mL) until the OD 600 nm reached 0.8-1.0. The cells were collected by centrifugation (5,000 rpm, 5 minutes) and washed twice with sterile PBS. The cells were then incubated in 100 ml of a monophasic Bligh-Dyer mixed solution at room temperature for 20 minutes. The mixed solution was centrifuged (2,000 g, 20 minutes), and the precipitate was washed twice with a monophasic Bligh-Dyer mixed solution. The insoluble residue was then resuspended with 27 ml of a sodium acetate buffer, pH 4.5 (containing 1% SDS), and boiled at 100° C. for 30 minutes. The mixed solution was added with 30 ml of chloroform and 30 ml of methanol, and centrifuged again (2,000 g, 20 minutes), and the lower phase was collected. The lower phase was washed twice with a pre-equilibrated biphasic Bligh-Dyer upper phase, and dried using a nitrogen dryer. The sample was finally dissolved in 100 μL of chloroform:methanol (9:1, v/v) for quantitative analysis. Equal volumes of lipid samples were mixed with 2,5-dihydroxybenzoic acid (Sigma-Aldrich, USA). The contents of lipid A and lipid A pEtN were determined using a Bruker Autoflex Speed MALDI system (Bruker, Germany) in a negative ion mode. The modification of the lipid A was quantified by calculating the peak intensity ratio of the lipid A (m/z 1796.2) to the lipid A pEtN (m/z 1919.2).

### II. STATISTICAL ANALYSIS

All data were derived from at least three independent duplicate experiments, and expressed as mean±standard deviation (SD). Statistical analysis was performed using GraphPad Prism 9.0 software. P values were utilized to determine statistical significance, with the specific thresholds: *P<0.05, **P<0.01, ***P<0.001, and ****P<0.0001. For comparison between two groups, an unpaired t test was used, while for comparisons of multiple groups, one-way analysis of variance (one-way ANOVA) was used. In an animal infection model, a Log-rank (Mantel-Cox) test was utilized to evaluate the significance of differences in overall survival rate.

### III. RESULTS

### 1. Synergistic Bactericidal Effect of Ini and Colistin in Eliminating Enterobacteriaceae Bacterium Carrying mcr In Vitro and Inhibiting Biofilm Formation

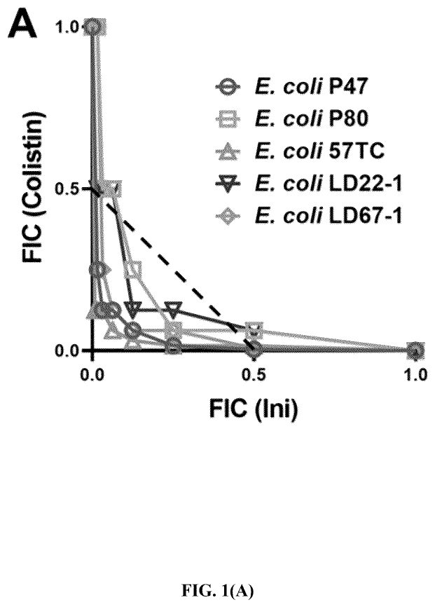

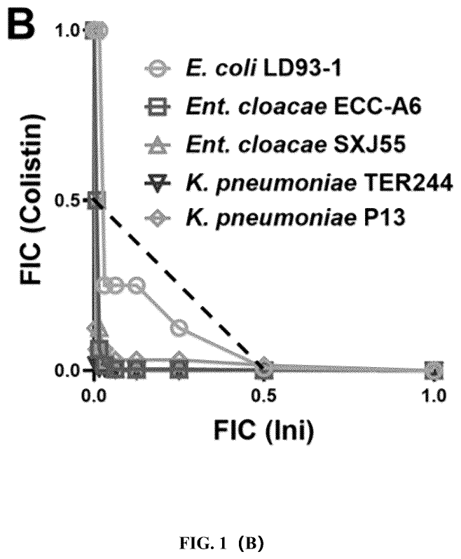

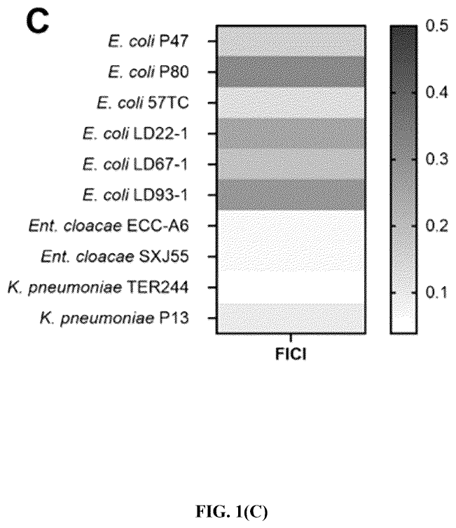

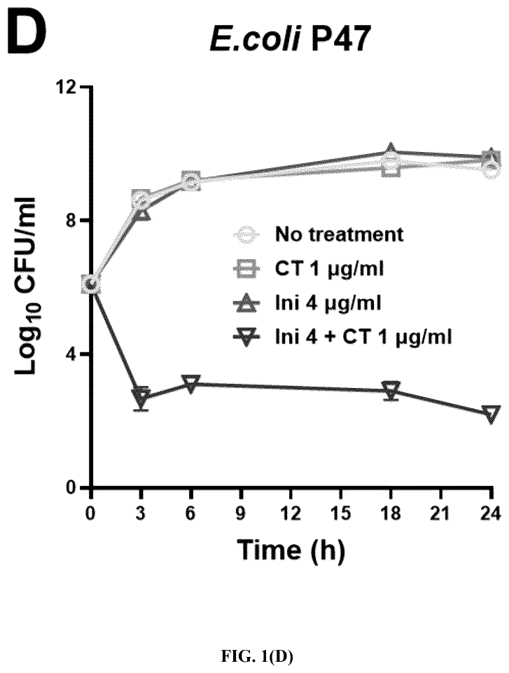

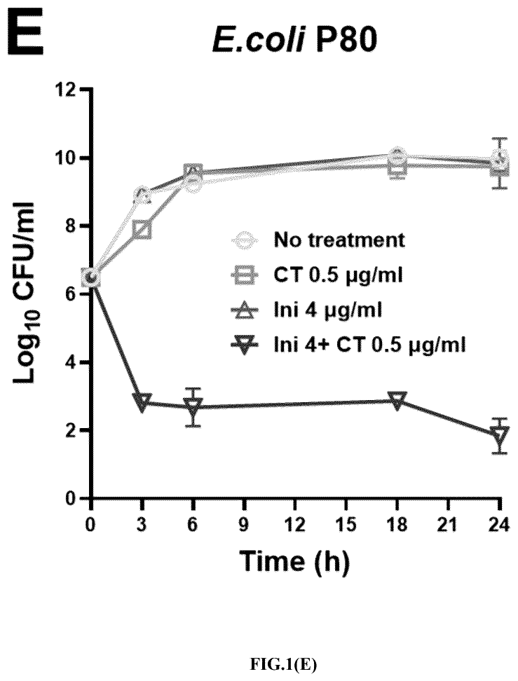

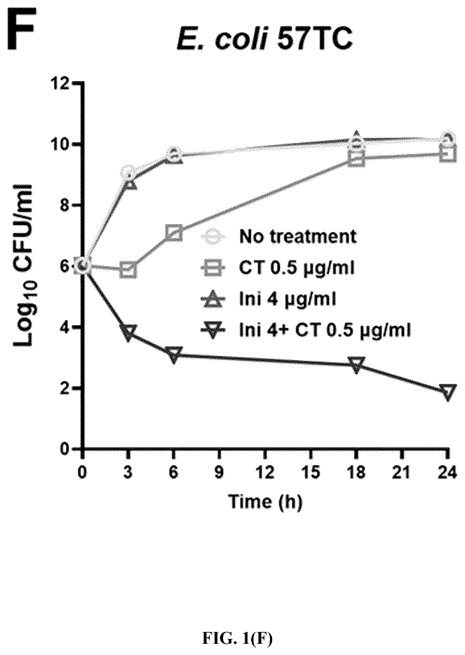

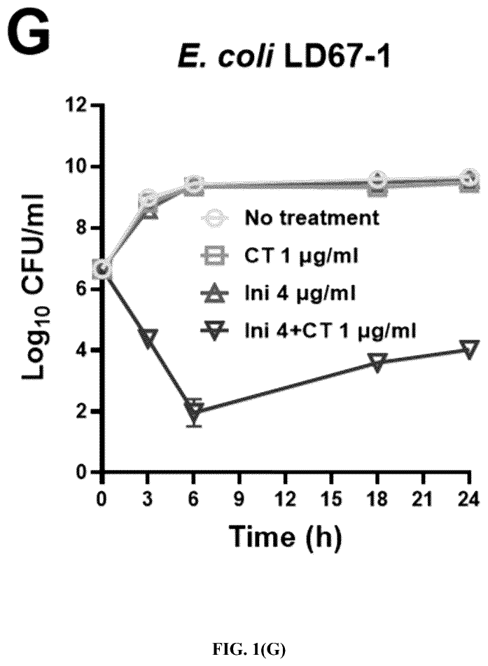

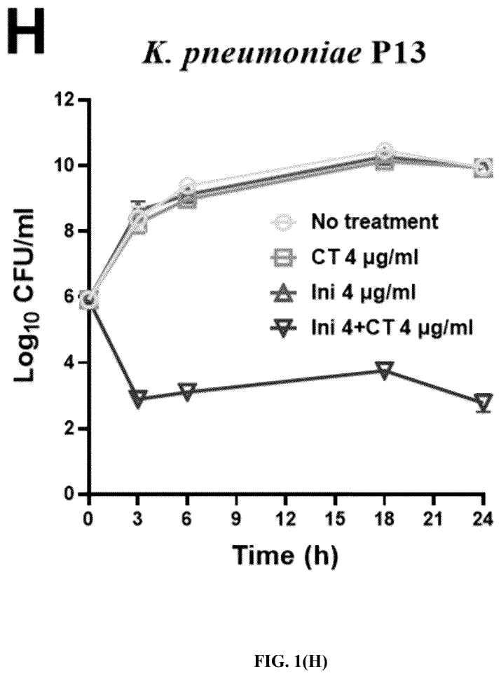

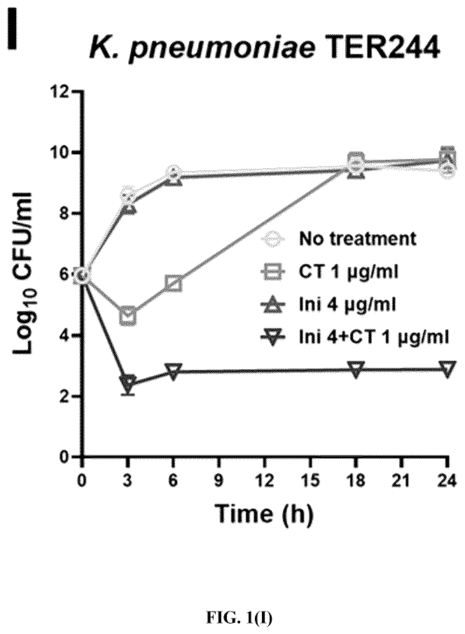

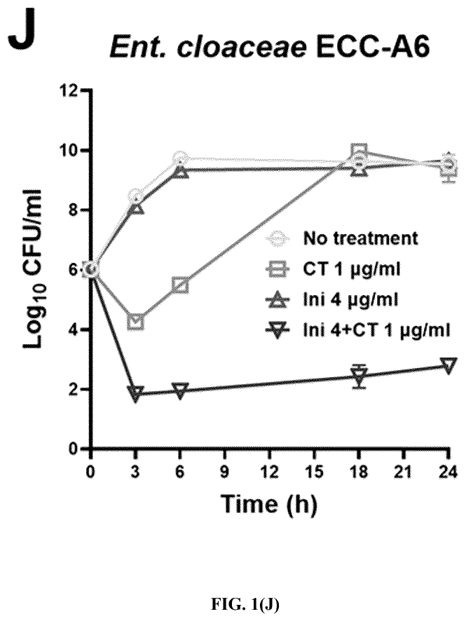

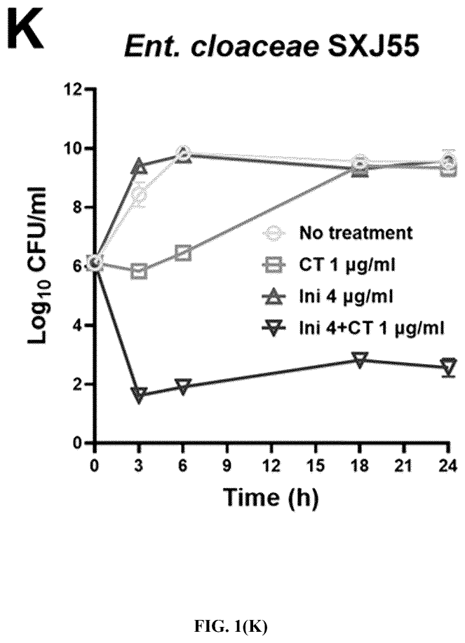

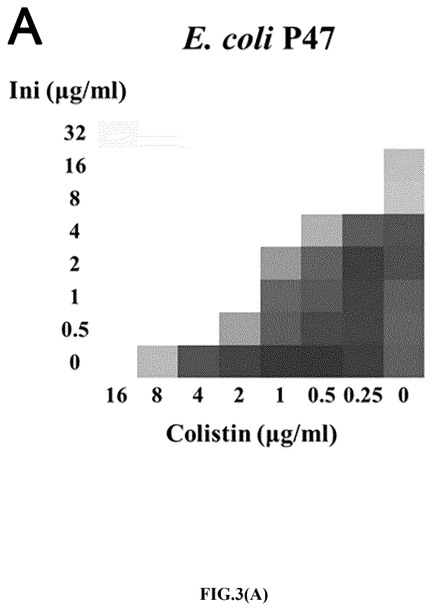

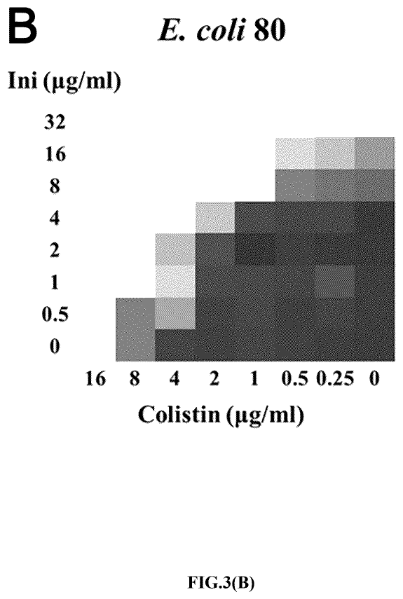

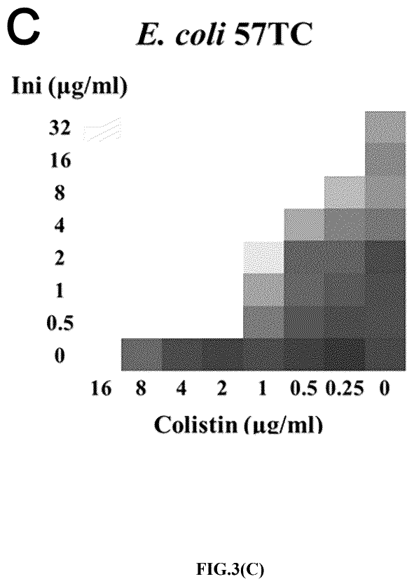

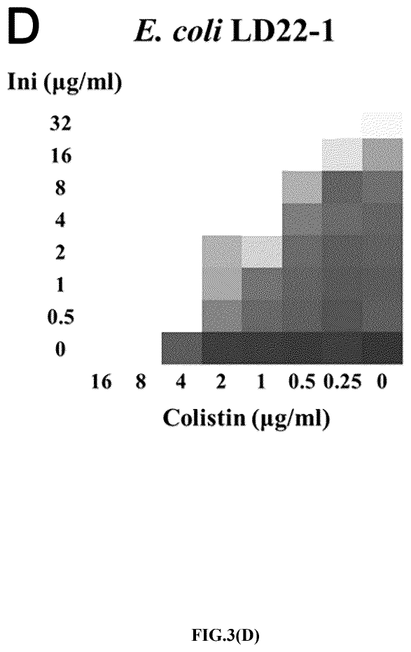

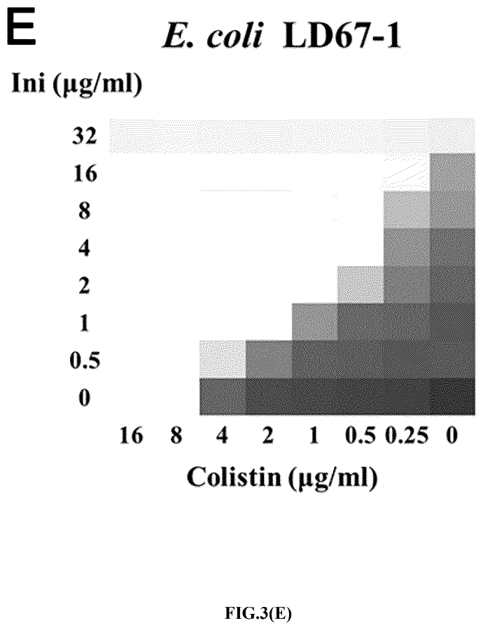

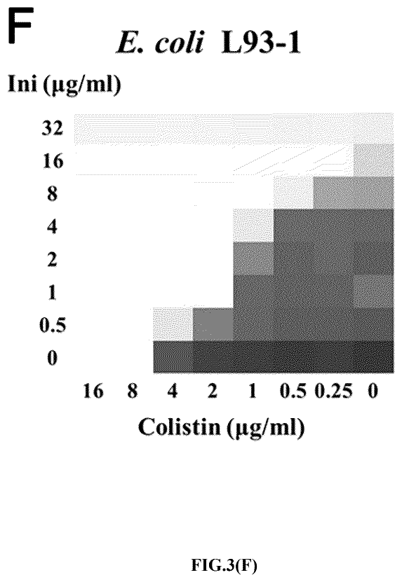

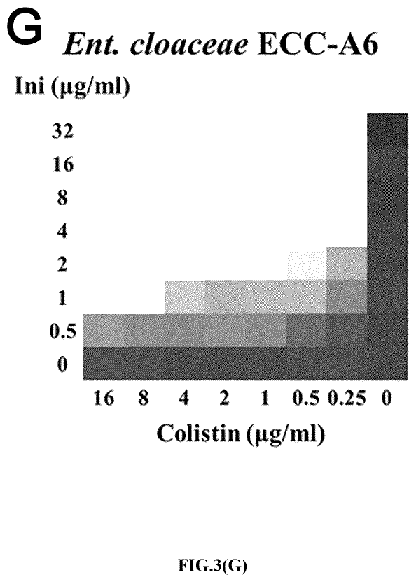

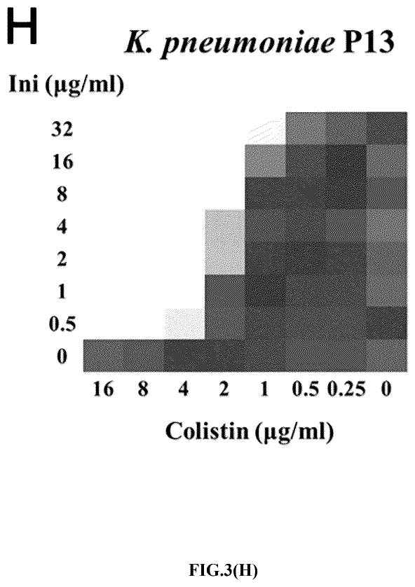

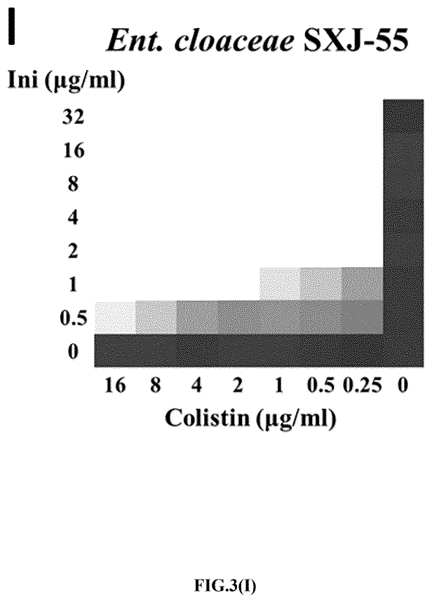

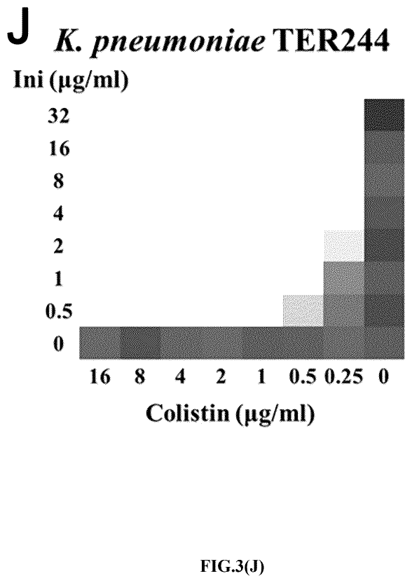

In the initial screening, a chessboard analysis method was employed to evaluate the synergistic effect of Ini and colistin in eradicating an Enterobacteriaceae bacterium carrying mcr (including Escherichia coli (n=6), Enterobacter cloacae (n=2), and Klebsiella pneumoniae (n=2). As shown in the contour maps and heat maps, a synergistic bactericidal effect was observed when Ini and colistin were used in combination, as evidenced by an FIC index below 0.5 (FIGS. 1A, 1B, 1C, and FIG. 3). The FIC indices for all strains were shown in Table 1 above.

The effect of the combined use of Ini and colistin in the treatment of the Enterobacteriaceae bacterium carrying mcr was further evaluated using a time-dependent bactericidal experiment (FIGS. 1D-1K). Ini alone could not inhibit the growth of most tested strains within 24 hours of culture. For Escherichia coli 57TC, Klebsiella pneumoniae TER244, Enterobacter cloacae ECC-A6 and Enterobacter cloacae SXJ55, colistin alone inhibited the growth of bacteria in the first 6 hours, but the inhibition action was weakened after prolonged treatment for 18 hours. In contrast, the combined use of Ini and colistin significantly reduced the bacterial load by approximately 10⁷ times within 24 hours of treatment, effectively eliminating almost all bacterial cells.

Given that infections caused by Enterobacteriaceae bacteria such as Escherichia coli were often associated with biofilm formation, inhibiting such a process could enhance the efficacy of antibiotics (reference: RömLing, U. & Balsalobre, C. Biofilm infections, their resilience to therapy and innovative treatment strategies. Journal of internal medicine 272, 541-561 (2012).).

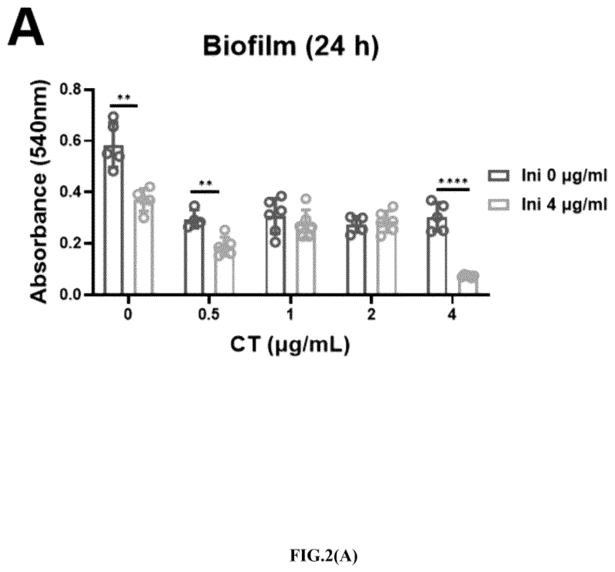

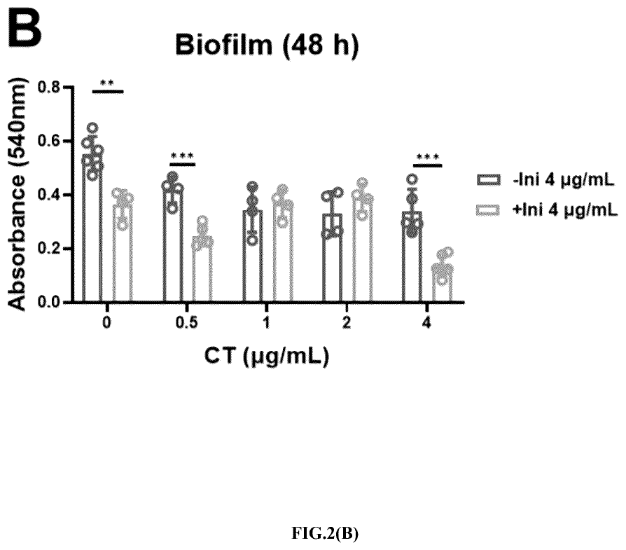

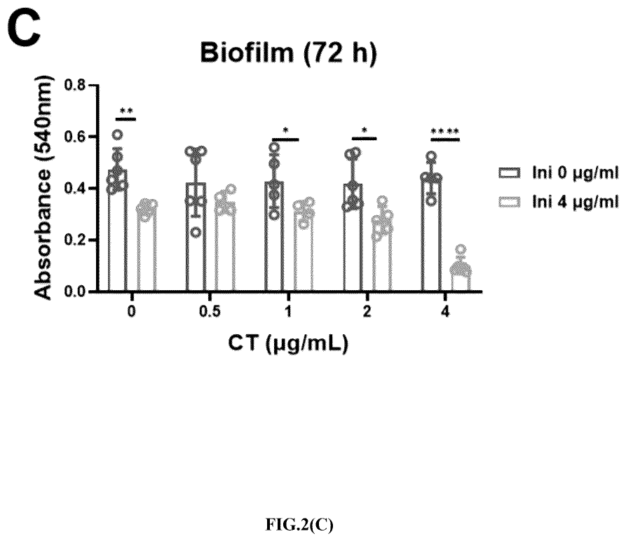

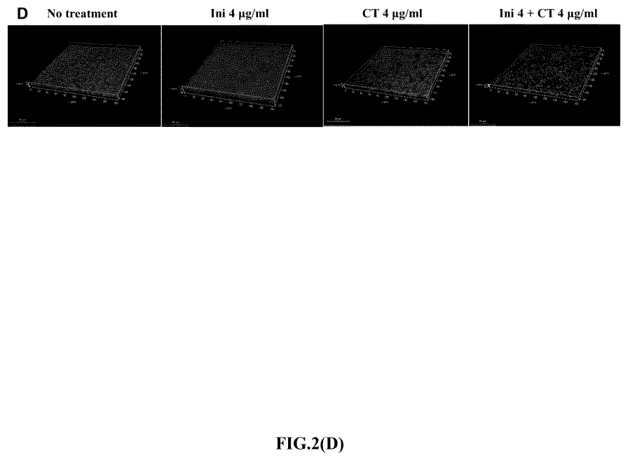

The present disclosure employed Propidium iodide (PI) staining to study whether Ini could restore and enhance the inhibitory effect of colistin on biofilm formation (FIGS. 2A, 2B, and 2C). Use of Ini alone could significantly inhibit the biofilm formation of Escherichia coli P47 at a sub-inhibitory concentration (4 μg/mL). Compared with colistin alone, the addition of 4 μg/mL of Ini could significantly enhance the inhibitory effect of colistin in a time-dependent manner. Similarly, the biofilms of bacteria after treatment with Ini, colistin, and a combination thereof were imaged using confocal laser scanning microscopy (CLSM), further validating the synergistic inhibitory effect. The CLSM image showed that the combined use of Ini and colistin significantly eliminated the biofilm structure compared with the use of either Ini or colistin alone (FIG. 2D).

In conclusion, the results indicated that Ini was a promising adjuvant capable of restoring and enhancing the activity of colistin in vitro.

### 2. Ini Restored the Bactericidal Effect of Colistin in Mammalian Cells and Mouse Infection Model

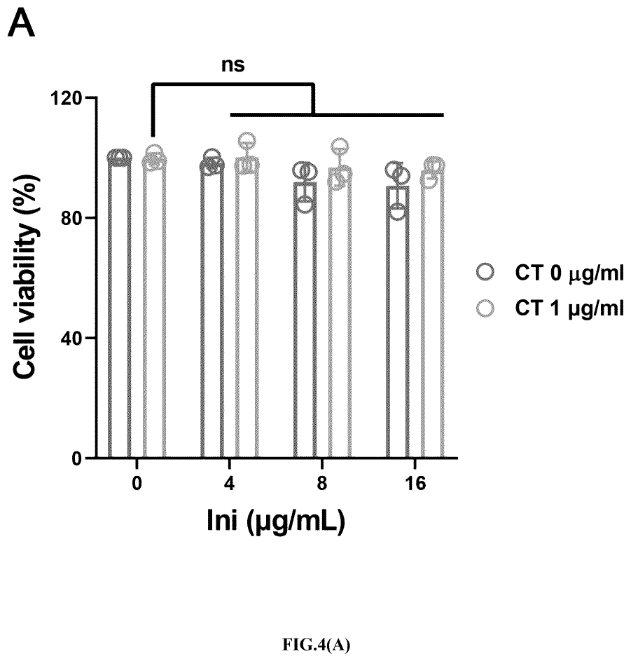

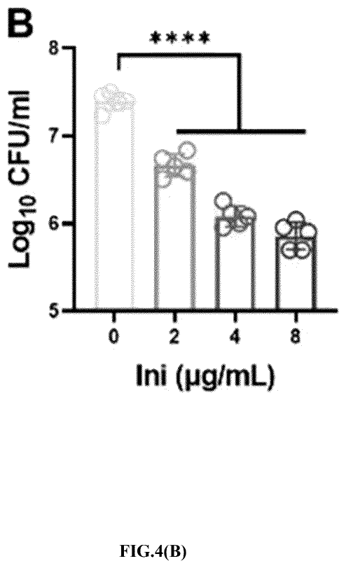

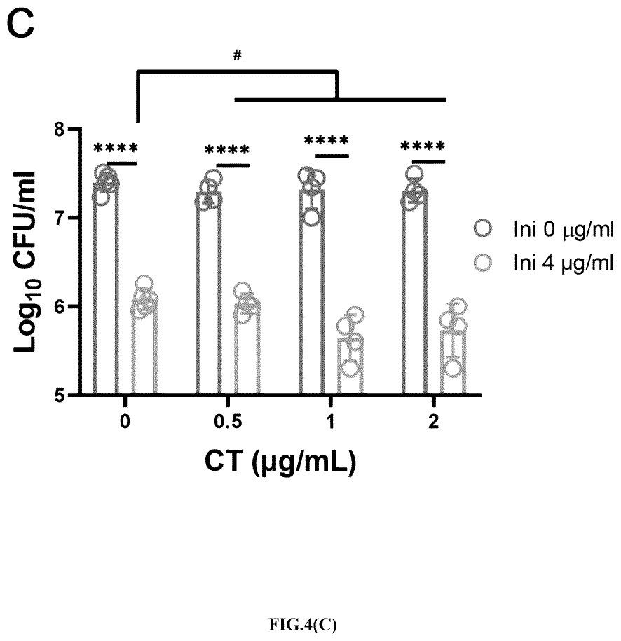

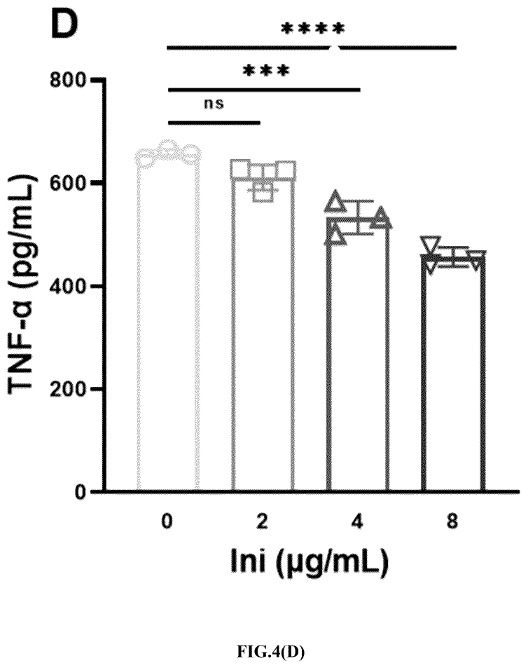

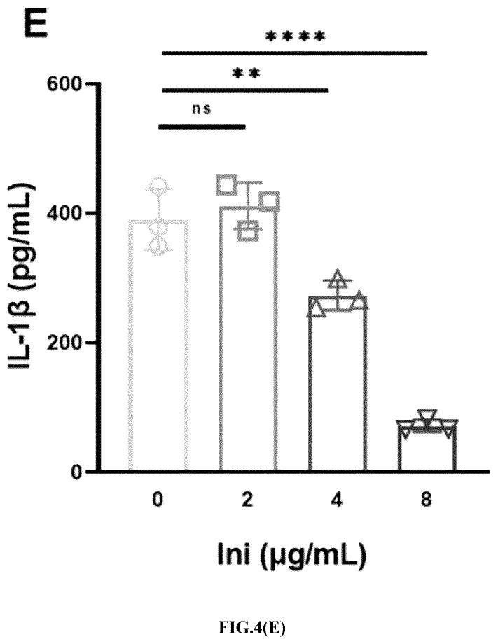

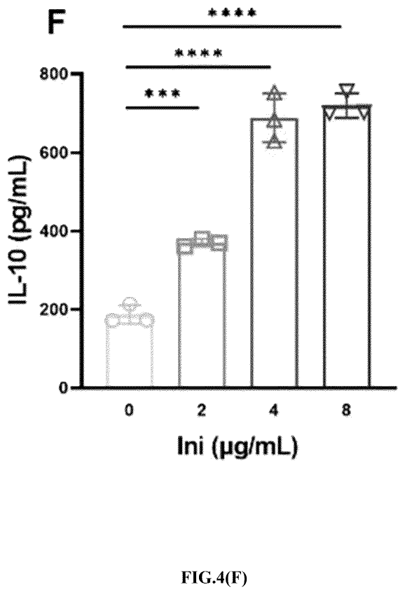

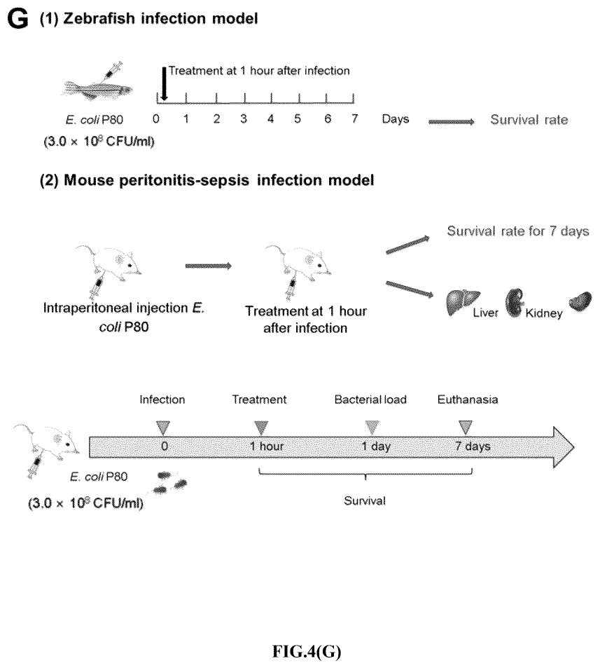

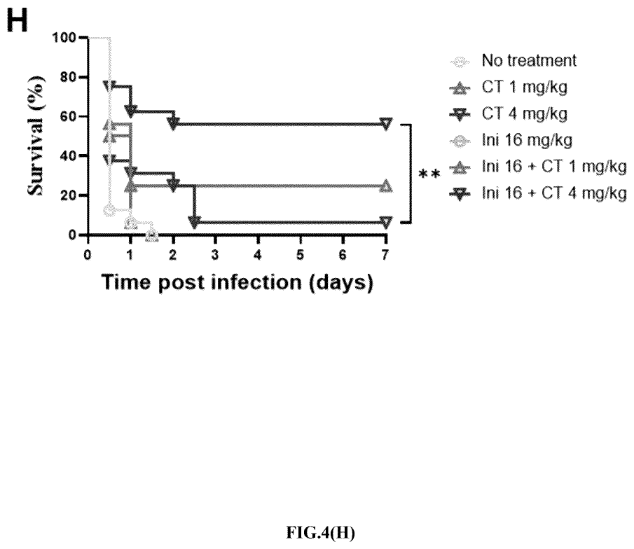

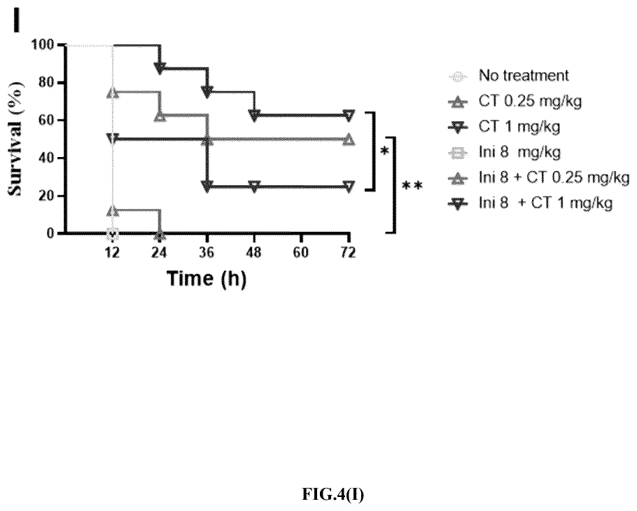

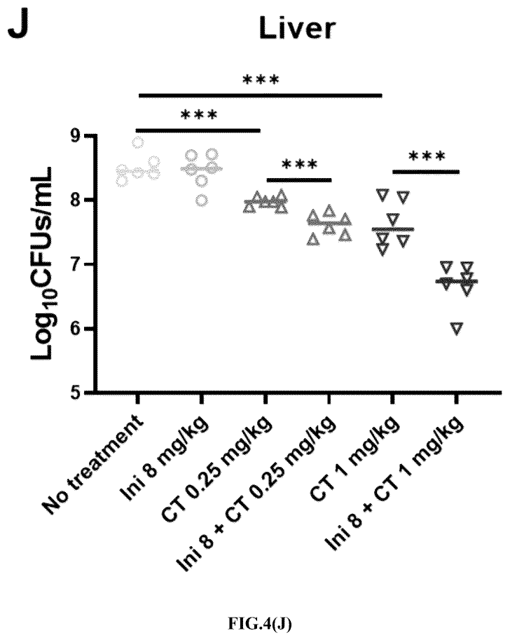

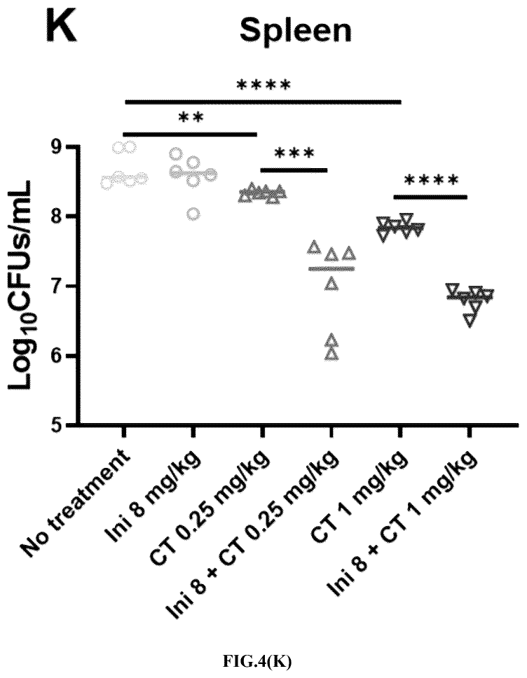

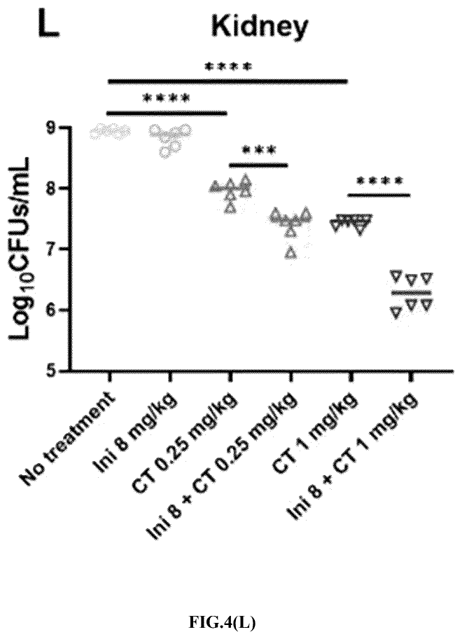

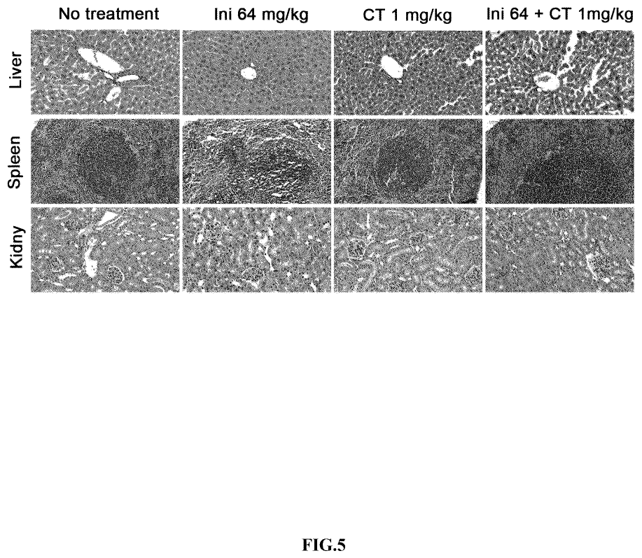

Considering that the role of Escherichia coli as a model organism in microbial research has been widely recognized, the present disclosure selects Escherichia coli P47 as a model strain for further research. In view of the synergistic bactericidal effect of Ini and colistin on the Enterobacteriaceae bacterium carrying mcr, the present disclosure evaluated the therapeutic effect of this combination on intracellular bacteria. First, the present disclosure used Vero cells as a model to evaluate the cytotoxicity of Ini and colistin on mammalian cells. The results showed that more than 93% of the Vero cells could survive after treatment with Ini, colistin, and a combination thereof, indicating that either Ini or colistin alone or a combination thereof had little cytotoxic effect on the mammalian cells (FIG. 4A). Furthermore, it had been shown by histopathological analysis that there were no obvious pathological changes in the liver, spleen, and kidney tissues of the mice treated with either Ini or colistin alone or a combination thereof. These findings further confirmed that, at working concentrations, neither Ini nor colistin monotherapy nor combined therapy thereof was toxic to mammalian organs (FIG. 5).

In the cell infection model, the ability of Ini to clean up intracellular bacteria was dose-dependent (FIG. 4B), while colistin at a concentration of 0.5-2 μg/mL had no obvious bactericidal effect. It was worth noting that, the addition of 4 μg/mL of Ini to colistin significantly reduced the bacterial load by 100 fold compared with colistin alone, indicating that the combined administration had a better antibacterial effect against intracellular pathogens (FIG. 4C).

Subsequently, the present disclosure explored the immunomodulatory property of Ini, as tetracycline drugs had been previously reported to downregulate LPS-induced cytokine production. This property could reduce excessive inflammatory responses and help prevent tissue damage. After stimulating RAW264.7 with LPS to induce inflammatory responses, the present disclosure treats the cells with different concentrations of Ini. The results showed that the production of the proinflammatory cytokines tumor necrosis factor-α (TNF-α) and interleukin-1β (1L-1β) was inhibited, while the production of the anti-inflammatory cytokine interleukin-10 (IL-10) was stimulated (FIGS. 4D-4F). This dual effect suggested that Ini not only could inhibit the excessive production of harmful pro-inflammatory mediators, but also could promote the release of protective anti-inflammatory factors. These immunomodulatory properties of Ini were crucial for alleviating tissue damage associated with excessive inflammation and may offer benefits to the treatment of inflammatory diseases and bacterial infections.

The present disclosure further evaluated how Ini affected the therapeutic effect on treatment of in vivo infection caused by Escherichia coli carrying mcr. In the zebrafish infection model (FIGS. 4G and 4H), zebrafishes were initially infected with 3×10⁸ CFU/ml of Escherichia coli P80. Untreated zebrafishes and zebrafishes treated with either colistin (1 mg/kg) or Ini (16 mg/kg) alone died due to infection within 36 hours. In contrast, combinatory therapy of Ini (16 mg/kg) and colistin (1 mg/kg) improved the survival rate, rescuing 25% of animals by 7 days post infection. Moreover, the survival rate for the combinatory therapy of Ini and colistin was significantly improved to 56.25%, compared with the survival rate of only 6.25% for colistin (4 mg/kg) monotherapy. Such obvious improvement indicated that Ini could significantly restore the bactericidal effect of colistin in the zebrafish infection model (p=0.0042).

To further evaluate the therapeutic effect, the present disclosure constructed a mouse peritoneal infection model (FIGS. 4G and 4I). After the mice were infected with 3×10⁸ CFU/ml of Escherichia coli P80, the mice treated with Ini monotherapy (4 mg/kg) died within 12 hours after infection, while the mice treated with colistin monotherapy (0.25 mg/kg) died within 24 hours after infection. Furthermore, the survival rate for the colistin monotherapy (1 mg/kg) was only 12.5%. In contrast, the combinatory therapy of 4 mg/kg of Ini and 1 mg/kg of colistin could significantly increase the survival rate to 62.5% (p=0.0303), highlighting the higher efficacy of the combinatory therapy.

### 3. Ini and Colistin had Synergistic Effect in Destroying Bacterial Membrane

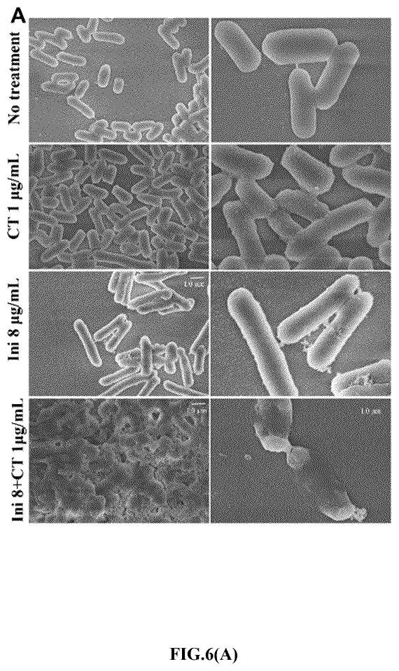

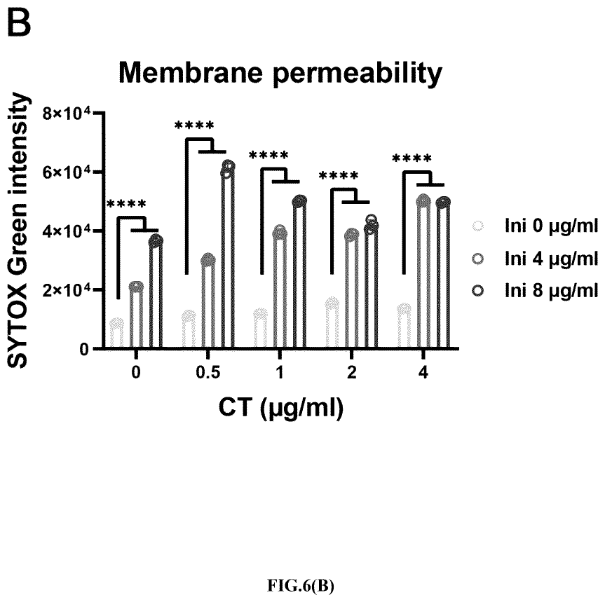

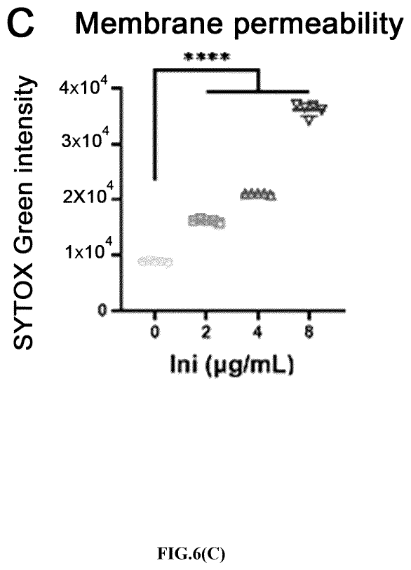

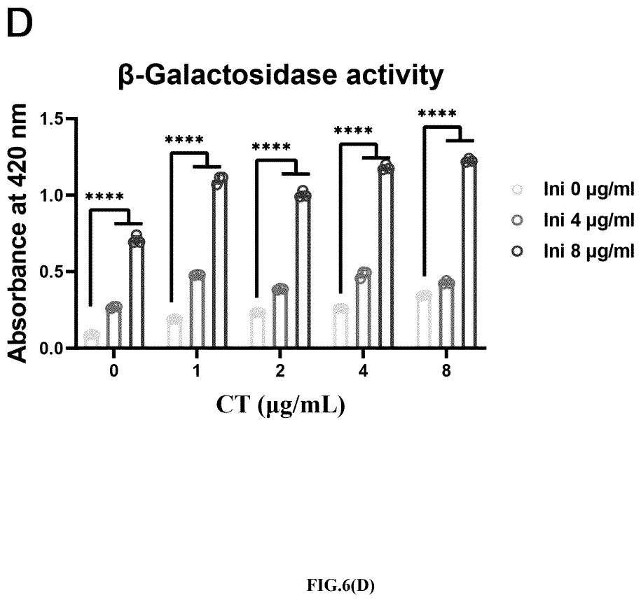

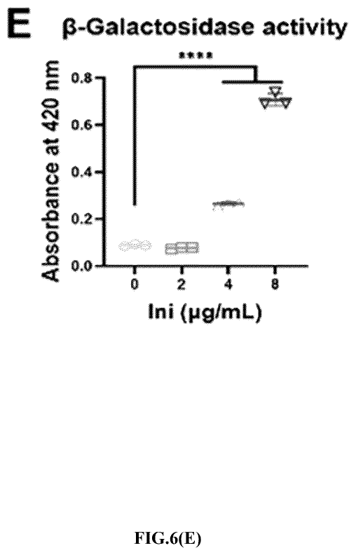

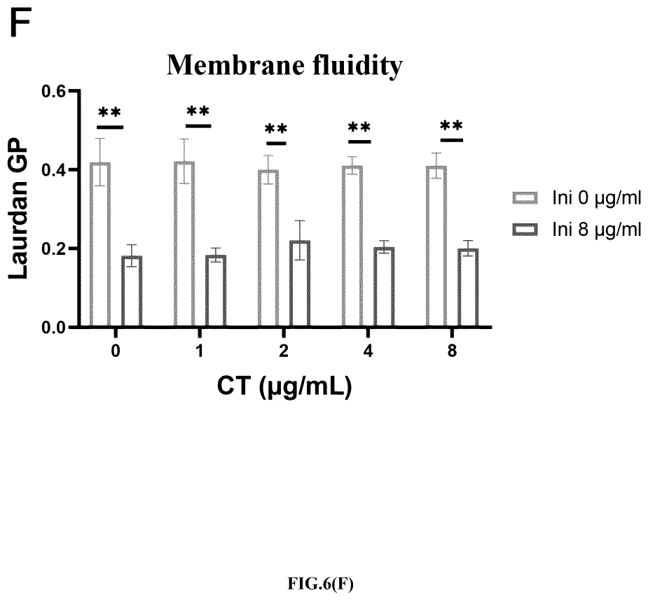

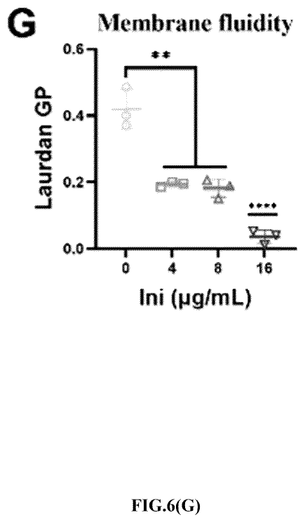

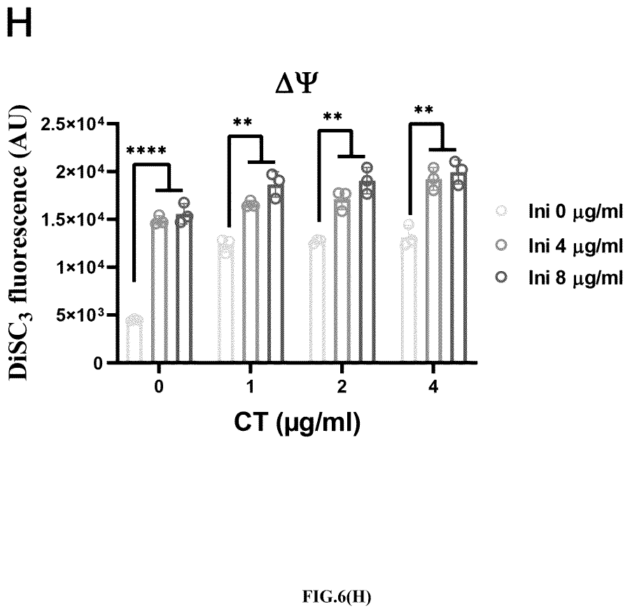

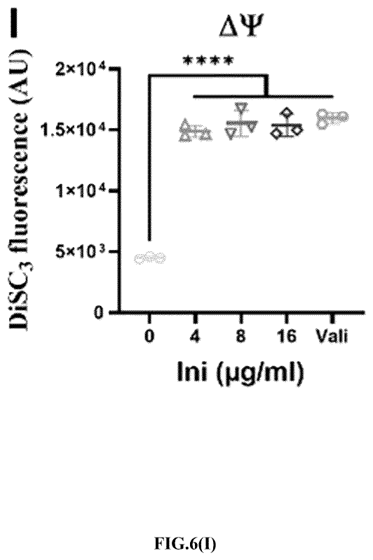

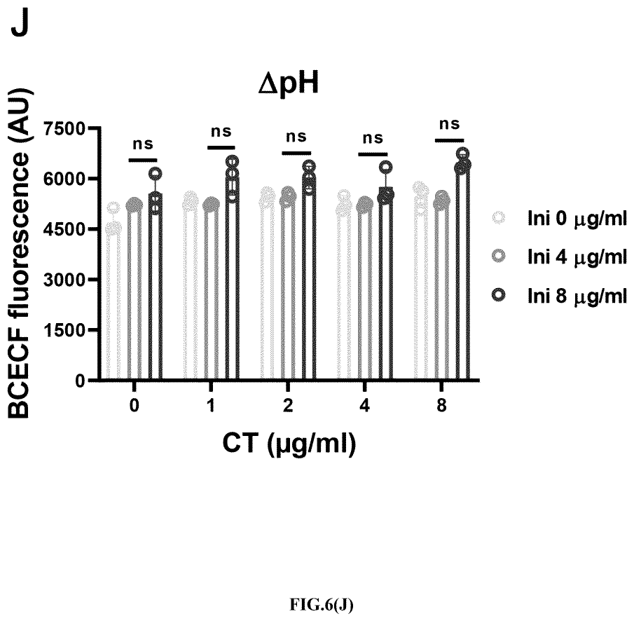

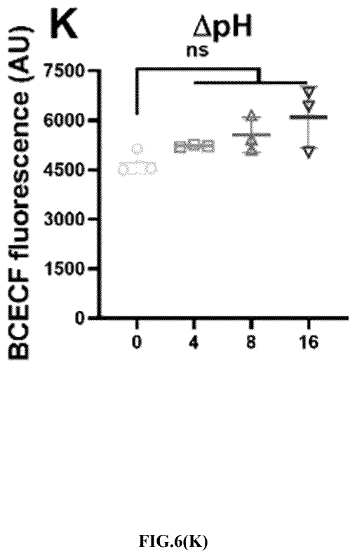

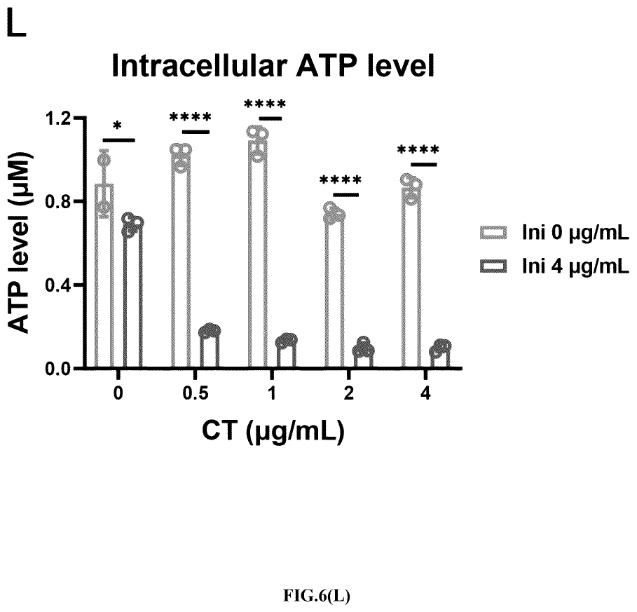

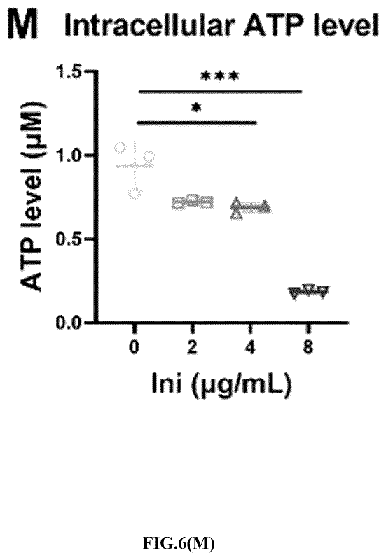

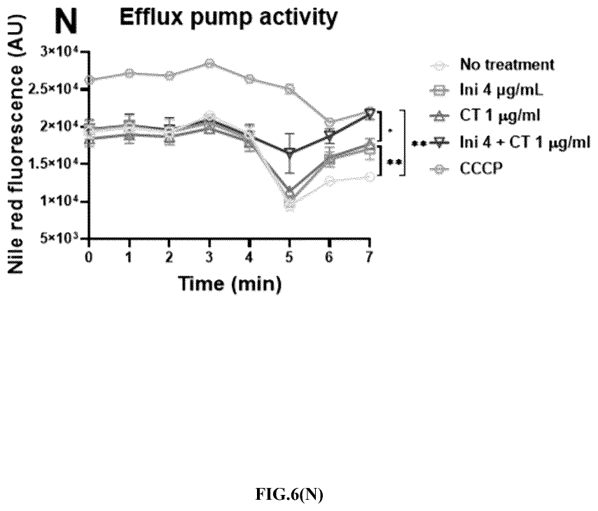

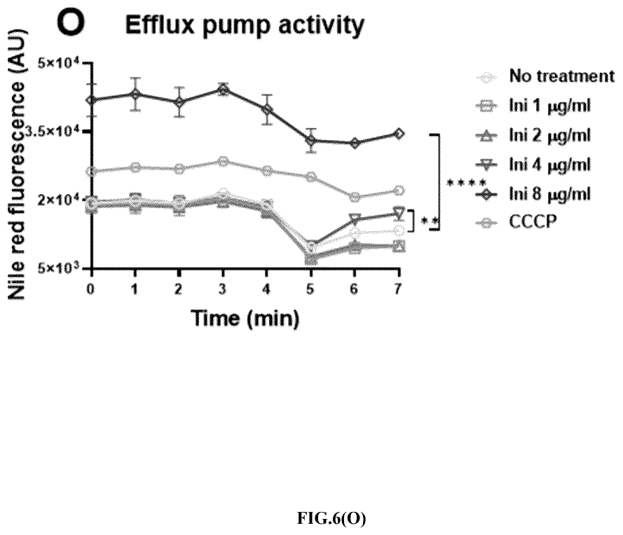

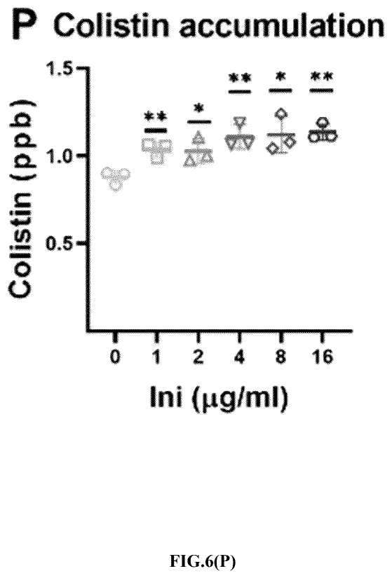

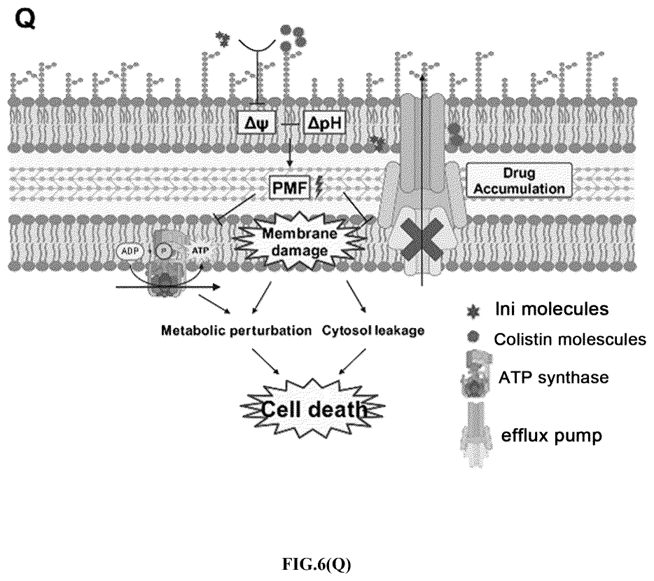

After proving the synergistic effect of Ini and colistin in eliminating the enterobacteria carrying mcr in vitro and in vivo, the present disclosure then observed the morphological changes of Escherichia coli P47 after being treated with Ini and colistin by scanning electron microscopy (SEM) (FIG. 6A). No obvious change in the bacterial morphology was observed when Escherichia coli P47 was treated with sub-inhibitory concentration of Ini (8 g/mL) or colistin (1 μg/mL) alone. The simultaneous use of 8 μg/mL of Ini and 1 μg/mL of colistin resulted in complete destruction of the bacterial cell membrane, manifested as clearly visible cell membrane shrinkage and rupture. Most cells showed signs of complete rupture, leading to leakage of cellular fluid and ultimately cell death.

Previous studies had shown that, the MCR enzyme present in the enterobacteria carrying the mcr gene could promotes specific modification of lipid A in the cytoplasmic membrane. These changes effectively reduced the ability of colistin to induce bacterial membrane penetration and disruption. In the present disclosure, the permeability of the bacterial membrane after treatment with Ini and colistin was evaluated preliminarily using SYTOX Green staining. The fluorescence of this fluorescent nucleic acid stain was enhanced when the bacterial membrane was penetrated, which could be used as an effective index of membrane integrity. Compared with colistin alone, the addition of Ini could significantly increase the intensity of SYTOX Green, indicating that Ini could significantly restore the membrane permeation of colistin (FIGS. 6B and 6C). Inner membrane permeability was also evaluated by measuring β-galactosidase activity, as this enzyme could cleave ONPG only after bacterial membrane permeabilization. Compared with Ini or colistin alone, the combined use of Ini and colistin could improve the activity of β-galactosidase. Furthermore, the present disclosure also evaluated changes in membrane fluidity after treatment with Ini and colistin (FIGS. 6D and 6E). After Ini was used in combination with colistin, the Laurdan generalized polarization (GP) value of the bacterial cell membrane decreased significantly, indicating that the presence of Ini could increase membrane fluidity (FIGS. 6F, 6G). Given that the overall increase in membrane fluidity might come from the dissipation of bacterial membrane potential, and considering that several proton motive force dissipators (e.g. CCCP) had been found to synergize with colistin to eliminate bacteria, the present disclosure proposed the hypothesis that Ini might also have the function of a proton motive force dissipator.

### 4. Ini Dissipated the Proton Motive Force of Bacteria

A proton motive force (PMF) consisted of two basic parts: transmembrane potential difference (ΔΨ) and transmembrane proton concentration gradient (ΔpH). This was because the probe accumulates in polarized bacterial cells, but was rapidly released into a culture medium, emitting fluorescence upon disruption of ΔΨ. As shown in FIGS. 6H and 6I, Ini had the ability to disrupt bacterial ΔΨ and synergized with colistin to disrupt ΔΨ. Moreover, the effect of combined treatment with Ini and colistin on the ΔpH of bacteria was evaluated by using a pH-sensitive fluorescent probe BCECF-AM (FIGS. 6J and 6K). After the addition of Ini, the bacterial ΔpH did not change significantly regardless of the presence or absence of colistin. These findings suggested that Ini neither affected ΔpH alone, nor regulated ΔpH together with colistin. In summary, the research results of the present disclosure showed that Ini acted as a proton motive force dissipator and enhanced the proton motive force dissipation effect of colistin.

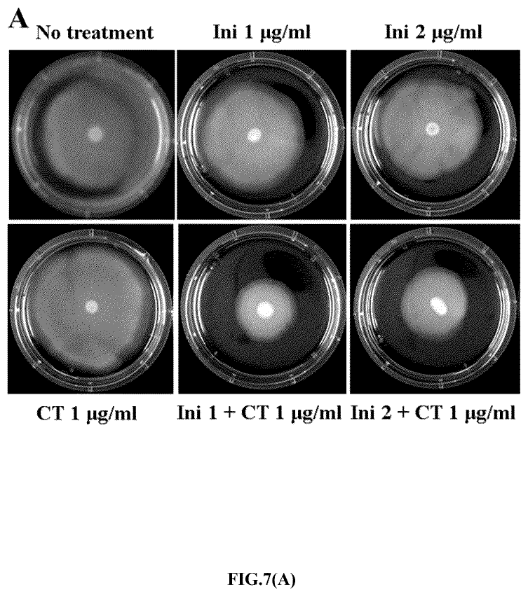

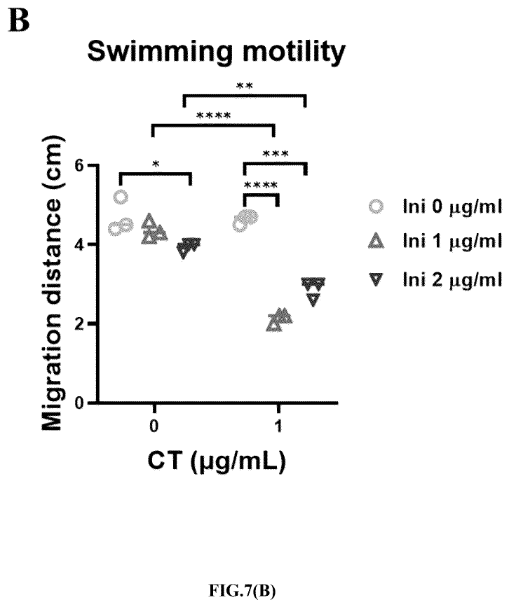

Given that the proton motive force driven various cellular processes, including adenosine triphosphate (ATP) synthesis, active transport of molecules, bacterial motility, and rotation of bacterial flagella, four functional assays were utilized to indirectly verify the dissipation of proton motive force caused by Ini and colistin. First, the present disclosure quantified the intracellular ATP level when Escherichia coli P47 carrying mcr was treated with Ini and different concentrations of colistin (0-4 μg/mL) simultaneously. The data showed that the response of the intracellular ATP level to colistin was significantly weakened after the addition of Ini, indicating that Ini had a synergistic effect on the activity of colistin in inhibiting ATP synthesis (FIGS. 6L, 6M). In the bacterial swimming test, Ini synergized with colistin to inhibit bacterial swimming. Compared with Ini alone or colistin alone, the migration distance of bacteria was significantly reduced when Ini (1-2 μg/mL) and colistin (1 μg/mL) were used in combination (FIG. 7). Nile red was a substrate for various efflux pumps in bacteria. Upon inhibition of these efflux pumps, Nile red accumulated inside bacterial cells, leading to increased intracellular fluorescence. As shown in FIGS. 6O and 6N, Ini inhibited the activities of the efflux pumps, indicating that it could also inhibit the efflux of colistin. This inhibition led to an increase in the intracellular concentration of colistin, thereby enhancing its antibacterial activity. In order to further verify these findings, the present disclosure quantified the intracellular concentration of colistin in bacterial cells treated with different concentrations of Ini (0-8 μg/mL). The results showed that the accumulation of colistin in bacterial cells increased significantly (FIG. 6P).

In summary, the research results of the present disclosure showed that Ini and colistin acted synergistically to effectively disrupt the proton motive force of bacteria. This disruption led to severe membrane damage and ultimately bacterial cell death (FIG. 6Q).

5. Ini could Inhibit the Adhesion and Invasion of Macrophages

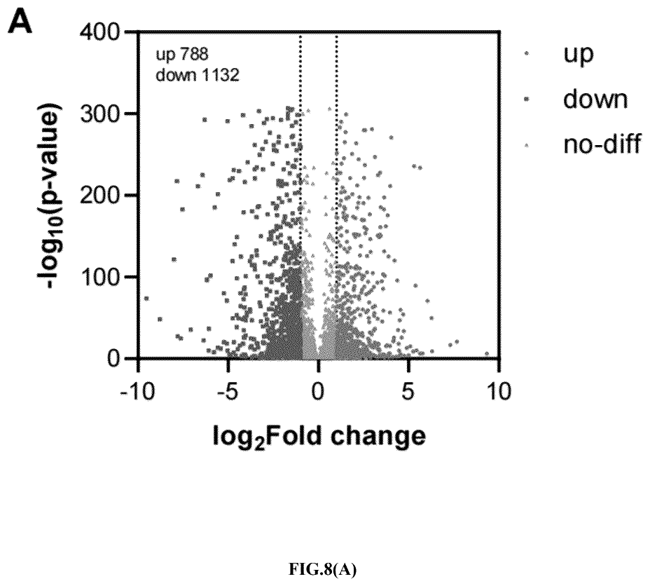

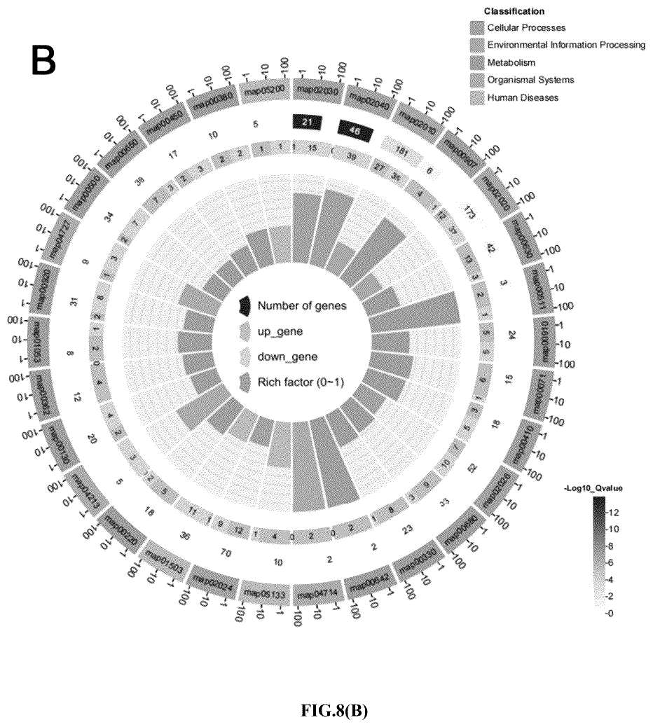

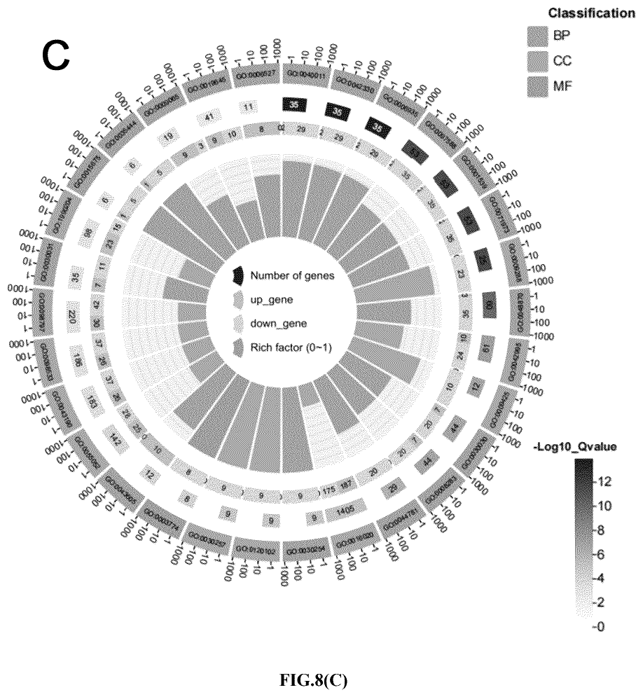

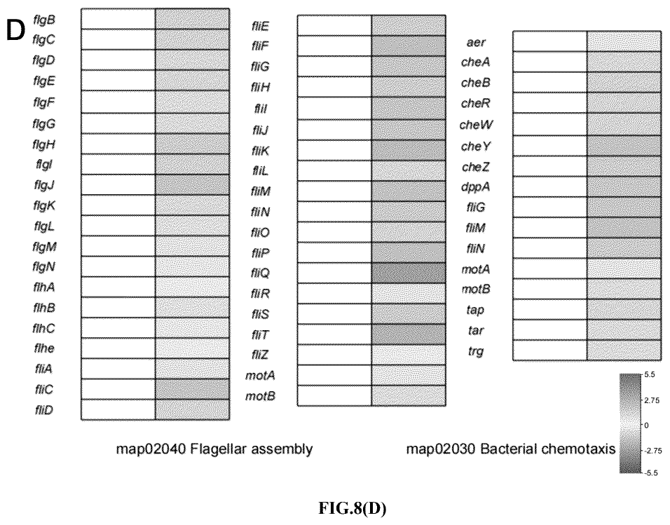

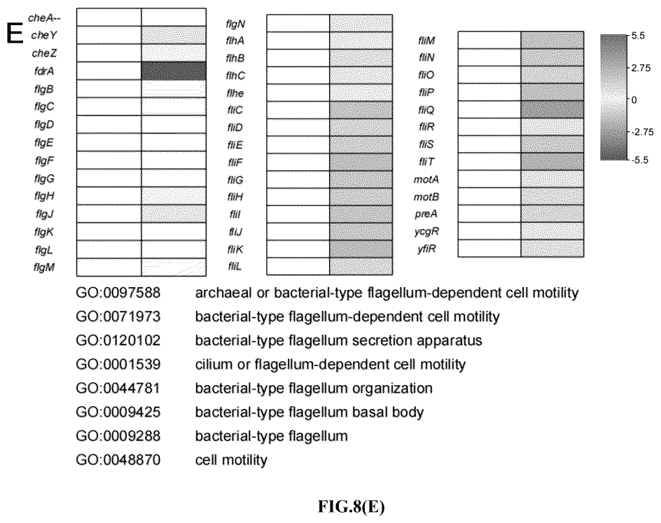

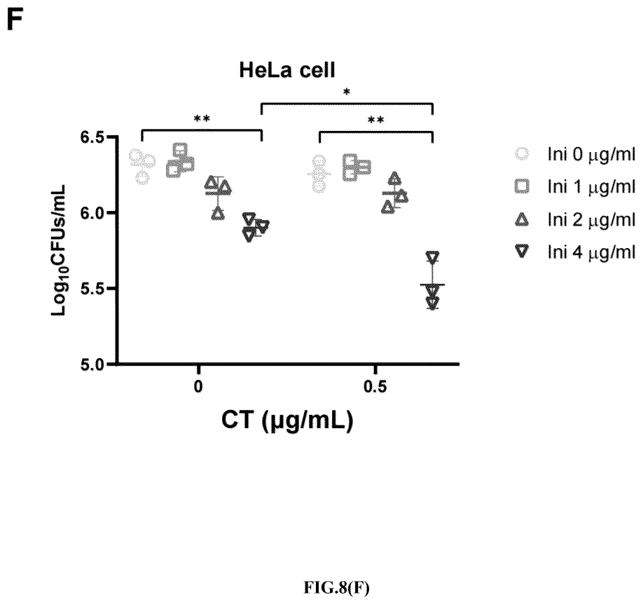

In order to elucidate the molecular mechanism of the synergistic bactericidal effect of Ini and colistin, the present disclosure conducted transcriptomic analysis on Escherichia coli P47 after colistin monotherapy or Ini and colistin combinatory therapy. The results showed that 507 differentially expressed genes (DEGs) were upregulated and 457 DEGs were downregulated respectively, when Ini and colistin combinatory therapy was used (FIG. 8A). Gene Ontology (Go) and Kyoto encyclopedia of genes and genomes (KEGG) enrichment analysis showed that, the DEGs were mainly involved in bacterial flagellar assembly, cell motility, and chemotaxis (FIGS. 8B and 8C). Specifically, as shown in FIGS. 8D and 8E, the DEGs involved in these biological processes showed a high degree of similarity. It was worth noting that, other than fdrA, yfiR and dppA, almost all DEGs were downregulated. This indicated that combined treatment with colistin and Ini could inhibit flagella formation and bacterial motility, which was consistent with the previous finding that Ini and colistin had a synergistic inhibitory effect on bacterial swimming mobility (FIG. 7). Flagella played a key role in multiple infection processes including biofilm formation, adhesion, and invasion. As shown in FIG. 8F, the addition of 4 μg/mL of Ini could significantly reduce the number of intracellular bacterial cells, indicating that Ini could inhibit bacterial adhesion and invasion to mammalian cells.

6. Ini Interfered with the Redox Process and Metabolism of Bacteria

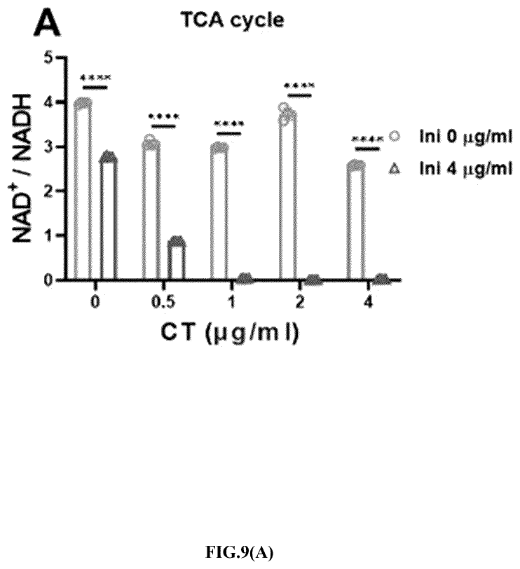

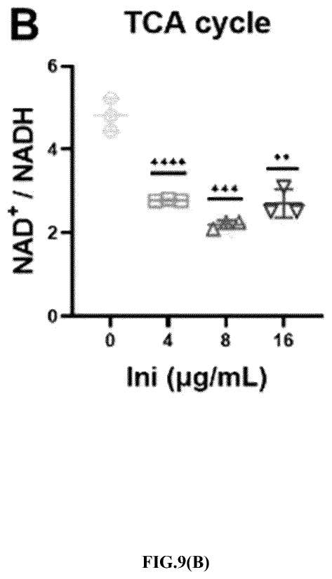

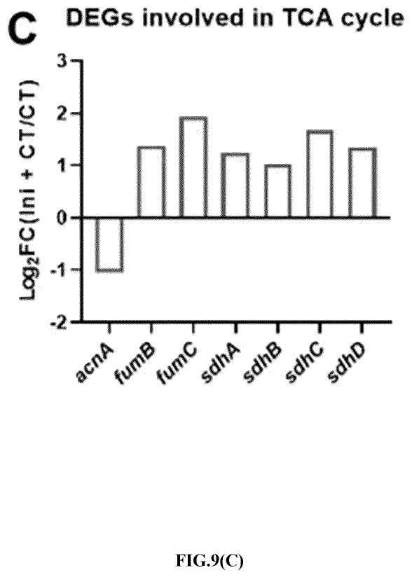

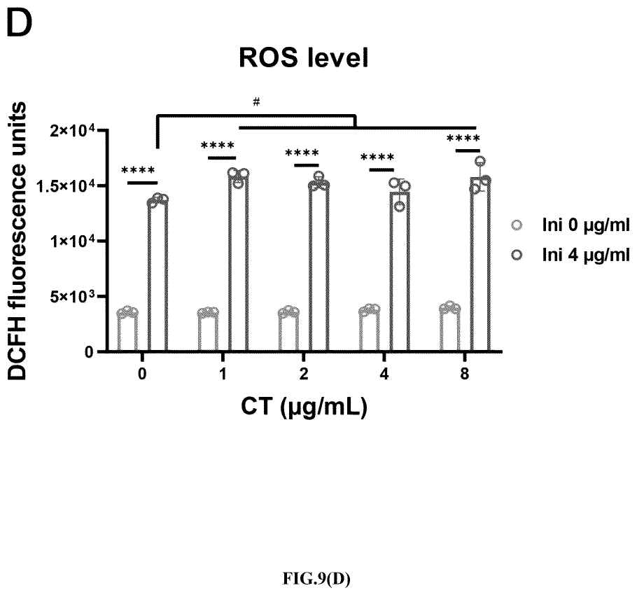

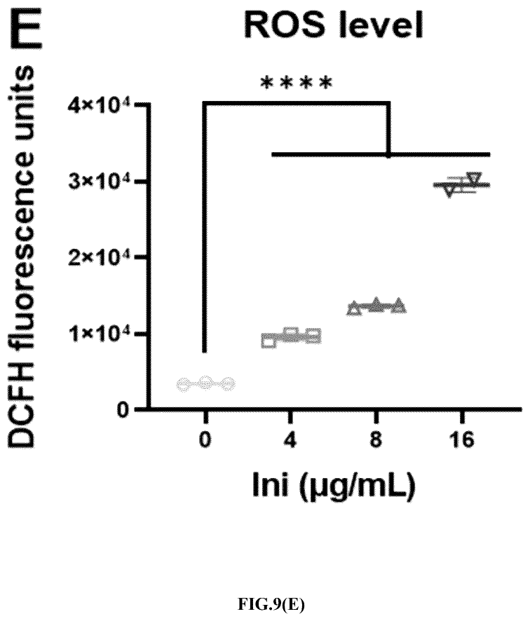

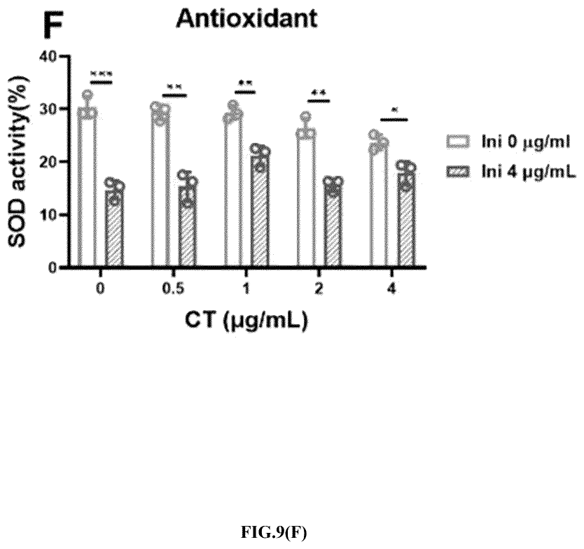

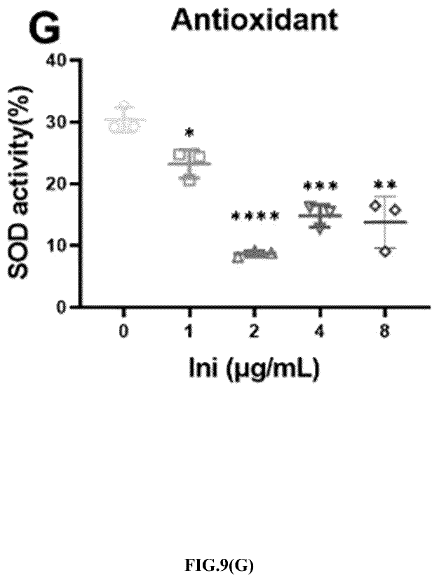

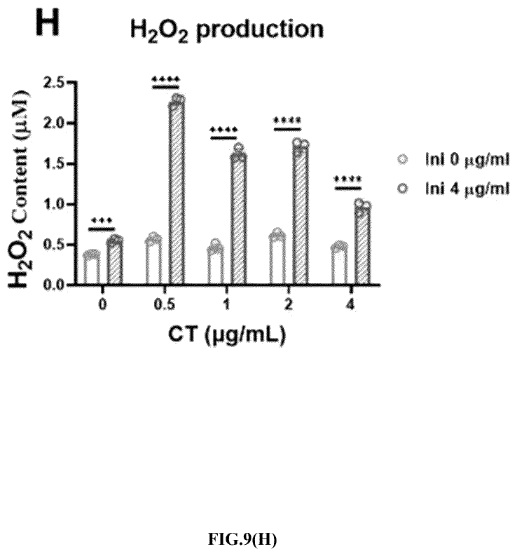

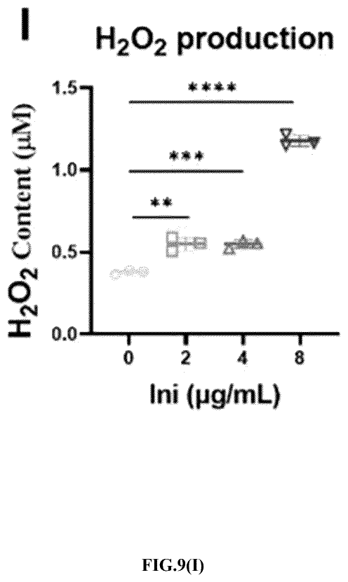

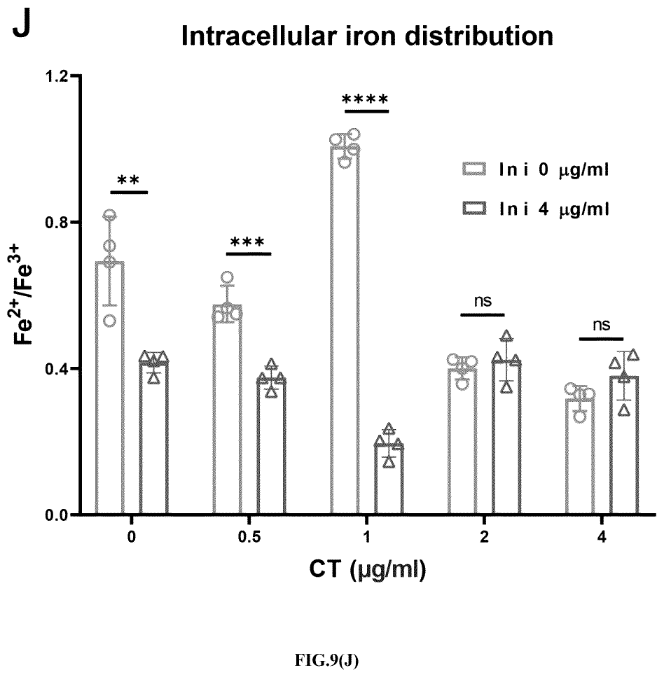

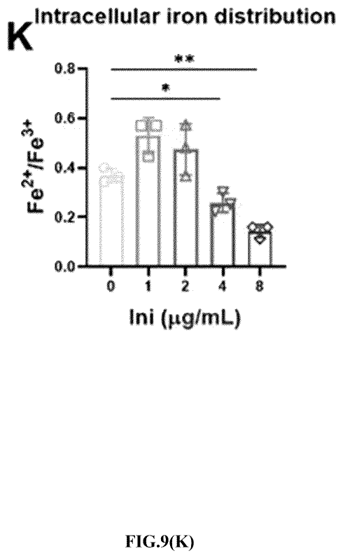

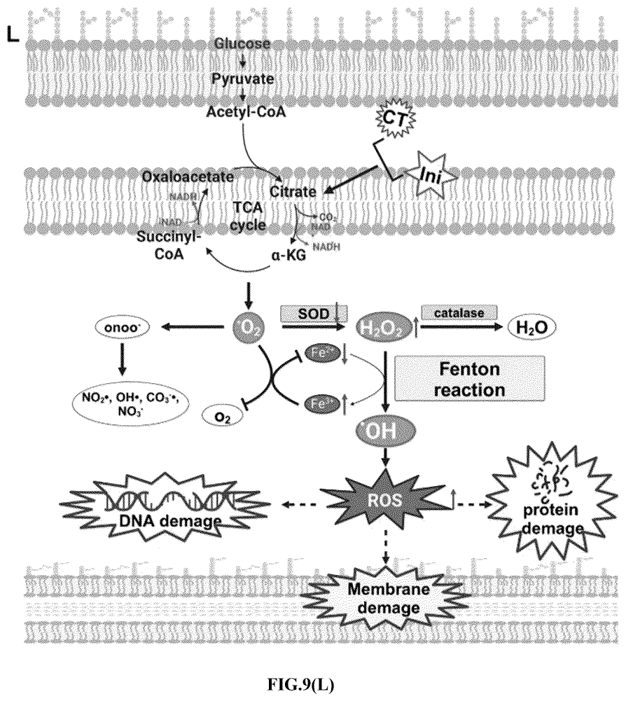

The synergistic bactericidal effect of Ini and colistin prompted the present disclosure to investigate whether the addition of Ini to colistin treatment would disrupt the redox process, as colistin generated ROS in gram-negative bacteria. First, the present disclosure determined the NAD/NADH ratio regulated by the TCA cycle, because the acceleration of the TCA cycle promoted the generation of ROS. Although using 4 μg/mL of Ini alone would not affect the ratio of NAD/NADH, the combined use of Ini and colistin would significantly reduce the ratio of NAD/NADH, which indicated that INI and colistin had a synergistic effect, accelerating the TCA cycle and inducing ROS production (FIGS. 9A, 9B). Compared with Escherichia coli P47 treated with colistin alone, transcriptome analysis of Escherichia coli P47 treated with a combination of Ini and colistin also showed that the expression levels of five of the six DEGs related to the TCA cycle were upregulated, including fumB, fumC, sdhA, sdhB, sdhC, and sdhD. Downregulation of acnA might inhibit the translation of AcnA, which appeared to be an enzyme that played a maintenance or survival role during an oxidative stress process. These results were consistent with the observed acceleration of the TCA cycle after the addition of Ini (FIG. 9C). In this regard, the addition of Ini increased ROS levels in both untreated bacterial samples and bacterial samples treated with colistin alone (FIGS. 9D, 9E). Moreover, superoxide dismutase (SOD) was a metalloenzyme that could catalyze the dismutation of superoxide radicals into oxygen molecules and hydrogen peroxide, the latter of which could be removed by catalase⁶⁸. Both Ini monotherapy and Ini and colistin combinatory therapy could significantly inhibit the activity of SOD in bacteria and increased the level of hydrogen peroxide, which indicated that Ini alone or Ini and colistin combinatory therapy both could promote the accumulation of ROS and induce oxidative stress damage in bacteria (FIGS. 9F, 9J, 9K, and 9I).

Furthermore, it was observed that Ini monotherapy or Ini and colistin combinatory therapy could reduce the intracellular Fe²⁺/Fe³⁺ ratio (FIGS. 9J, 9K). Consistent with the redox imbalance induced by the Fenton reaction, elevated hydrogen peroxide was converted to highly reactive hydroxyl radicals (—OH), during which ferrous irons were oxidized to ferric irons.

In view of the above, addition ODM Ini into colistin treatment could promote the generation and accumulation of ROS, leading to oxidative damage to DNA, proteins, and bacterial cell membranes (FIG. 9L).

### 7. Ini Inhibited Expression of Mcr and Modification of Lipid A

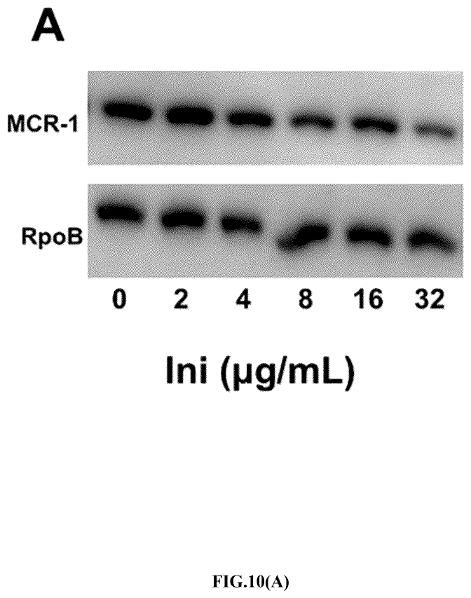

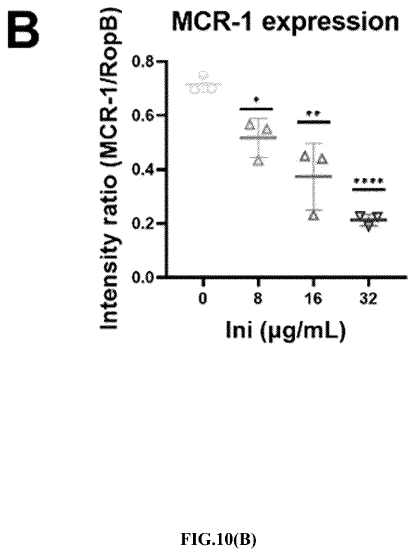

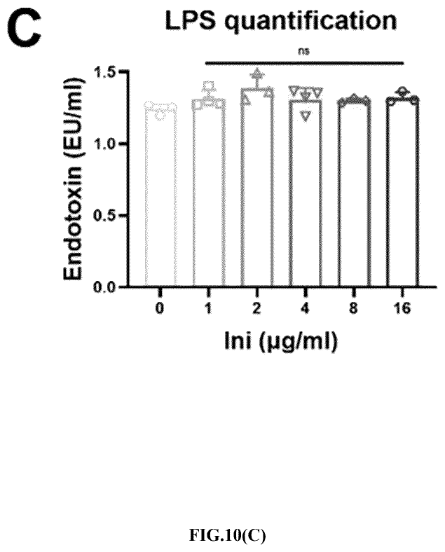

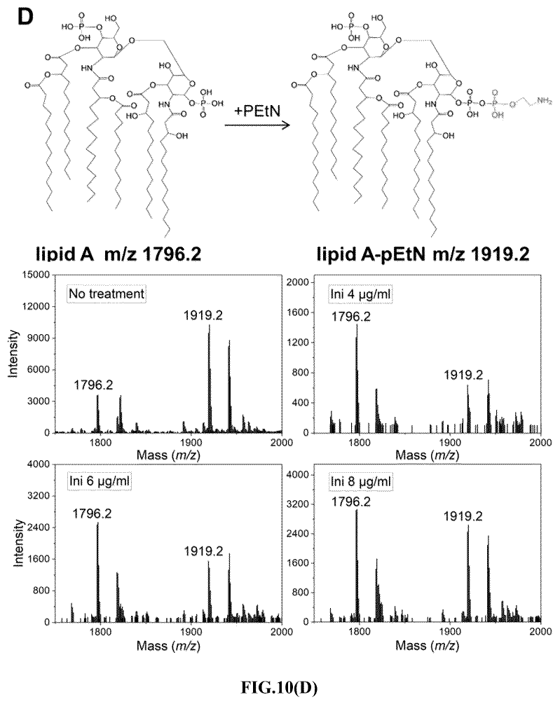

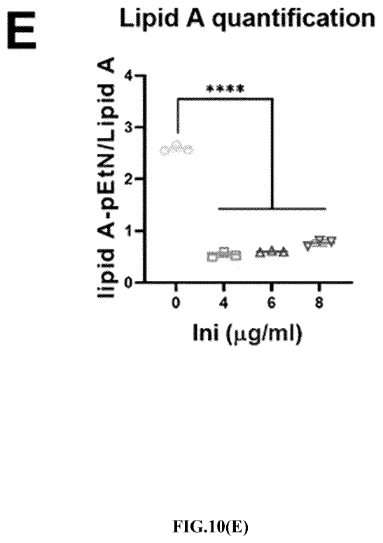

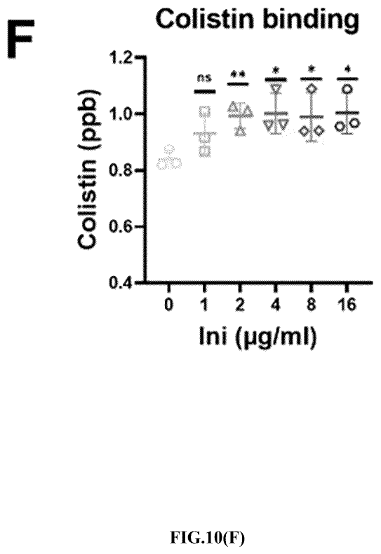

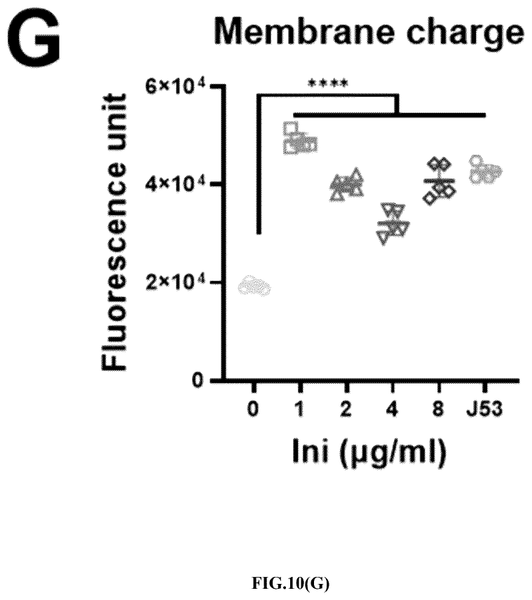

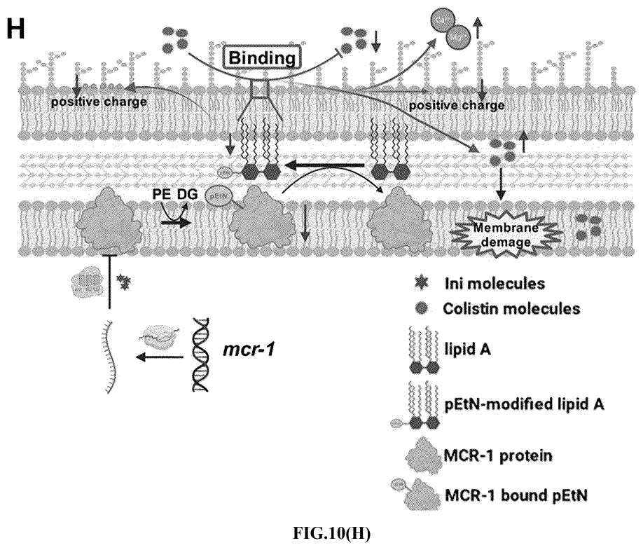

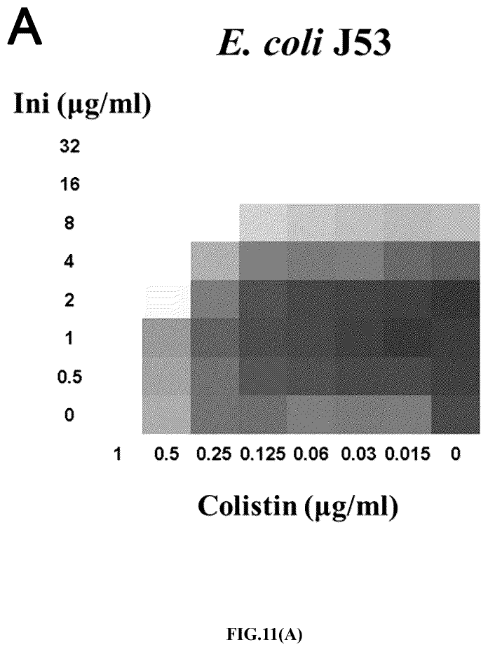

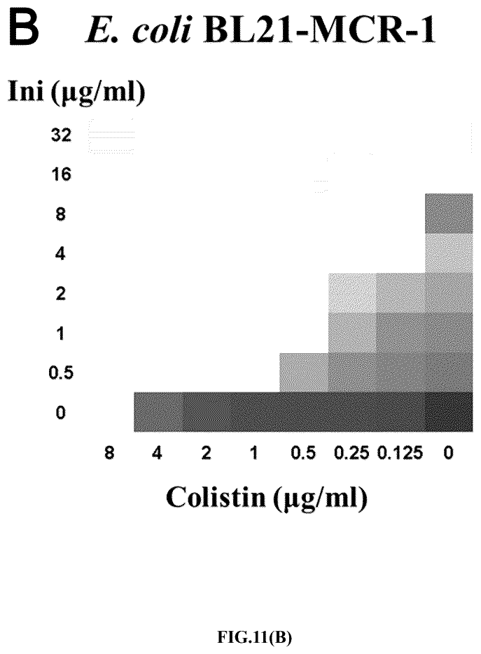

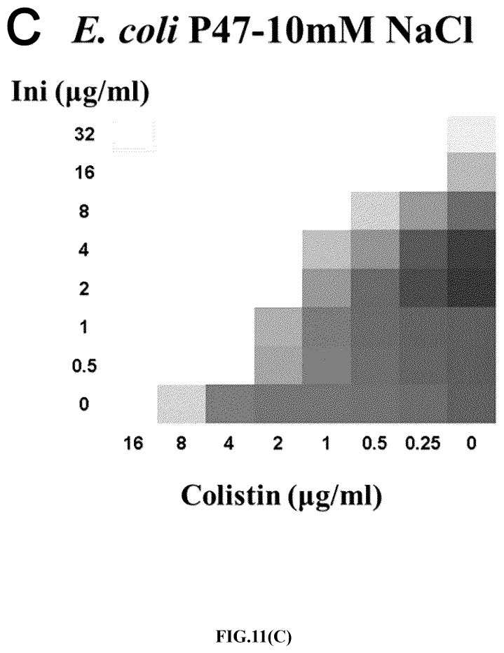

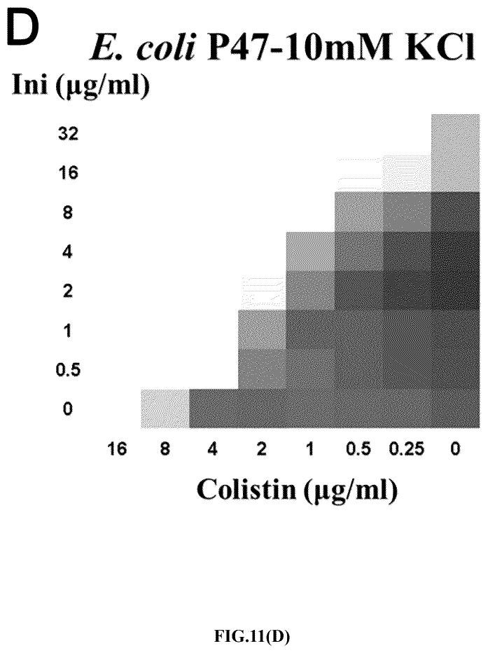

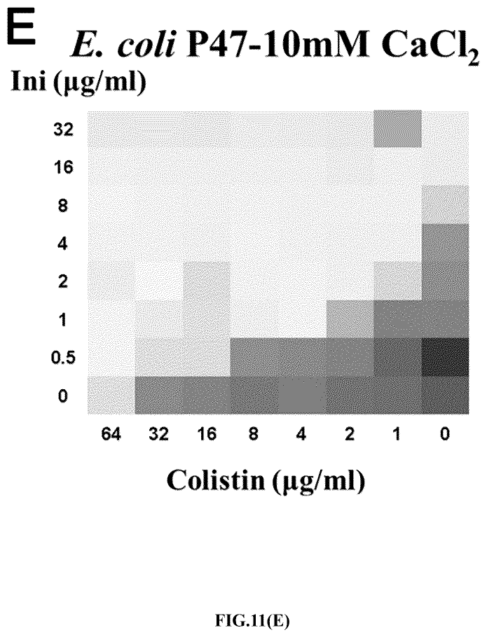

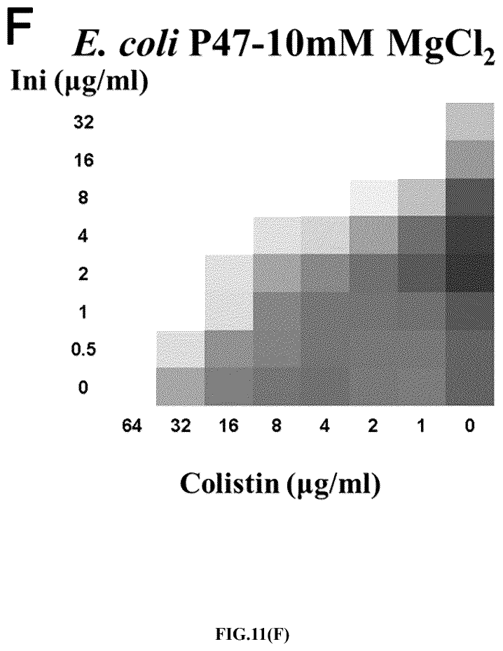

Given that the expression of mcr changed lipid A by adding phosphoethanolamine (pEtN) groups, leading to the decrease of sensitivity to Escherichia coli, the expression levels of mcr-1 after treatment with different concentrations of Ini were determined. The results showed that Ini could significantly inhibit the expression level of mcr-1 in Escherichia coli BL21-MCR-1 in a dose-dependent manner. Therefore, Ini restored the bactericidal effect of colistin on the strain, with an FIC index value of 0.1875 (FIGS. 10A, 10B, and 11). Similarly, when Escherichia coli J53 was treated with Ini and colistin, the FIC index was 0.75, indicating that no synergistic effect was observed (FIG. 10). After demonstrating that the addition of Ini did not affect LPS synthesis (FIG. 10C), in the present disclosure the proportion of modified lipid A (lipid A-phosphoethanolamine) was quantified. First, in the present disclosure MALDI-TOF mass spectrometry was utilized to obtain the mass spectra of lipid A and lipid A-phosphoethanolamine, and it had been found that their masses were 1796.2 Da and 1919.2 Da, respectively (FIG. 10D). The changes in lipid A after the addition of Ini were then quantified by calculating the peak intensity ratio of lipid A and lipid A-phosphoethanolamine. As expected, the proportion of lipid A-phosphoethanolamine was significantly reduced in the presence of Ini at concentrations of 4 to 8 μg/mL (FIG. 10E). Subsequently, after the addition of Ini, the amount of colistin bound to the bacterial cell membrane increased significantly, indicating that the inhibitory effect of Ini on lipid A modification could restore the affinity of colistin to lipid A (FIG. 10F). Moreover, the present disclosure also used FITC-labeled polyL-lysine (PLL) to determine membrane charges. After the addition of Ini, the fluorescence intensity was increased, indicating that Ini increased the positive charges on the bacterial cell membrane (FIG. 10G). In conclusion, Ini inhibited the modification of lipid A by inhibiting the expression of mcr, thereby restoring the binding of colistin to an outer membrane (FIG. 10H).

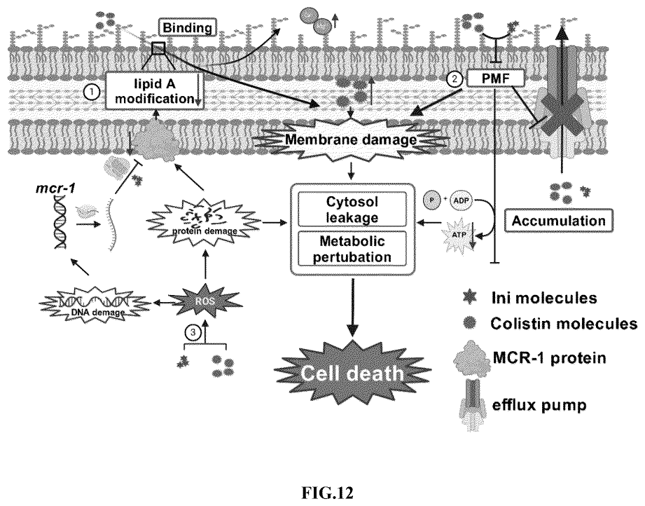

In summary, Ini restricted mcr expression, thereby inhibiting lipid A modification and promoting the entry of colistin into the bacterial cell membrane. Furthermore, Ini dissipated the PMF, which impaired the function of PMF-driven efflux pumps and led to the accumulation of colistin within the membrane. The accumulated colistin induced oxidative stress and exacerbated membrane damage, ultimately leading to cell death (FIG. 12).

### IV. CONCLUSION

In summary, the study of the present disclosure showed that Ini could enhance the therapeutic effect of colistin on the Enterobacteriaceae bacterium carrying mcr in both an in vitro experiment and an in vivo infection model (including zebrafish and mouse models). Mechanistic studies had shown that Ini inhibited the modification of lipid A by inhibiting the expression of mcr, thereby promoting the absorption of colistin by the bacterial cell membrane. Moreover, Ini could also dissipate the proton motive force and impair proton motive force-dependent efflux pumps, leading to the accumulation of colistin in the membrane. Therefore, the accumulation of colistin induced oxidative stress and membrane damage, ultimately leading to cell death (FIG. 12). The discovery of Ini as a novel colistin adjuvant provided a promising treatment method for the increasingly serious infection with Enterobacteriaceae bacteria carrying mcr.

The aforementioned description is only preferred embodiments of the present disclosure, rather than limiting the present disclosure, and any modification, equivalent substitution and improvement within the spirit and principle of the present disclosure should be included in the claimed scope of the present disclosure.

What is claimed is:

1. Use of incyclinide synergized with colistin in preparation of a bactericidal drug for an Enterobacteriaceae bacterium carrying an mcr gene.

2. The use according to claim 1, wherein the Enterobacteriaceae bacterium comprises Escherichia coli P47 and Escherichia coli P80.
